(19) United States
(12) Patent Application Publication
(10) Pub. No.: US 2023/0110568 A1
(43) Pub. Date: Apr. 13, 2023

(54) CRYOTHERAPY, THERMAL THERAPY, TEMPERATURE MODULATION THERAPY, AND PROBE APPARATUS THEREFOR

(71) Applicant: Monteris Medical Corporation, Minnetonka, MN (US)

(72) Inventors: Mark Andrew Grant, Winnipeg (CA); Brooke Ren, Maple Grove, MN (US); Richard Tyc, Winnipeg (CA)

(21) Appl. No.: 17/804,364

(22) Filed: May 27, 2022

Related U.S. Application Data
(63) Continuation of application No. 15/148,949, filed on May 6, 2016, now abandoned.
Application No. 15/148,949 is a continuation-in-part of application No. 14/841,109, filed on Aug. 31, 2015, now Pat. No. 10,327,830.
(60) Provisional application No. 62/141,612, filed on Apr. 1, 2015.

(51) Int. Cl. A61B 18/02

(52) U.S. Cl. CPC A61B 18/02; A61B 2018/0293; A61B 2018/0262; A61B 2018/00577

(57) ABSTRACT

In one aspect, recording instruments, probes, probe sheaths, and probe sleeves may include one or more recording elements, such as one or more ECG wires, EEG wires, and/or SEEG wires. A recording element may be used for lesion localization and assessment at the time of cryotherapy, thermal therapy, or temperature modulation therapy. A recording element may be used to provide positioning and monitoring during functional neurosurgery; to apply local tissue stimulation responsive to detection of an abnormal event to regulate cellular behaviors during treatment; to effect deep brain stimulation during a neurosurgical operation; to monitor internal electrical signals and identify abnormalities. Recording instruments may be deployed in vivo for hours or days while monitoring and analyzing signals. For signal analysis, leads disposed between recording element contact surfaces and along a shaft of the recording instrument may deliver recorded signals to a controller external to the patient for analysis.

### RELATED APPLICATIONS

The present application is a continuation-in-part of and claims the priority of U.S. patent application Ser. No. 14/841,109 to Grant et al. entitled “Cryotherapy, Thermal Therapy, Temperature Modulation Therapy, and Probe Apparatus Therefor” and filed Aug. 31, 2015, which is related to and claims the priority of U.S. Provisional Patent Application No. 62/141,612 to Grant et al. entitled “Small Diameter Probe” and filed Apr. 1, 2015, the contents of each of which are hereby incorporated in its entirety.

### BACKGROUND

Tumors, such as brain tumors, may be treated by heat (also referred to as hyperthermia or thermal therapy). In particular, it is known that above 57° C. all living tissue is almost immediately and irreparably damaged and killed through a process called coagulation necrosis or ablation. Malignant tumors, because of their high vascularization and altered DNA, may be more susceptible to heat-induced damage than normal tissue. Various types of energy sources may be used, such as laser, microwave, radiofrequency, electric, and ultrasound sources. Depending upon the application and the technology, the heat source may be extracorporeal (i.e., outside the body), extrastitial (i.e., outside the tumor), or interstitial (i.e., inside the tumor). One example treatment of a tissue includes interstitial thermal therapy (ITT), which is a process designed to heat and destroy a tumor from within the tumor itself. In this type of therapy, energy may be applied directly to the tumor rather than passing through surrounding normal tissue, and energy deposition can be more likely to be extended throughout the entire tumor.

Further, tumors and other abnormal cellular masses may be treated using a cryosurgical or cryotherapy technique where extreme cold conditions are applied to damage or destroy tissue. In one example, a coolant, such as liquid nitrogen or liquid argon, may be circulated within a probe device (cryoprobe) while in contact with tumorous tissue to freeze tissue within the vicinity of the cryoprobe.

### SUMMARY

In one aspect, the present disclosure relates to a variable length probe apparatus having a variable length probe structure including a probe and an adjustable depth stop to facilitate access to both shallow and deep targeted tissue areas. The variable length probe apparatus may be configured to accommodate lesions located at varying depths by repositioning over the adjustable depth stop. The probe portion, in some embodiments, is connected to an umbilical sheath for carrying inputs and outputs (e.g., energy, control signals, cooling gas or fluid, and/or heating gas or fluid) between the probe and a control unit. A transitional part configured for ease of grasping and manipulation of the probe may be disposed at the junction of the probe and the flexible umbilical sheath. The adjustable depth stop may be configured to connect to or otherwise rest upon a probe follower for remote rotational and/or linear positioning of the variable length probe apparatus and to prevent inadvertent extension into tissue beyond a treatment area.

In one aspect, the present disclosure relates to temperature modulation probes configured for modulated application of thermal therapy and cryotherapy using at least one thermal therapy-generating element as well as at least one cryotherapy-generating element disposed within the temperature modulation probe. In use, the temperature modulation probe supplies a modulated temperature output pattern to a target tissue, varying between at least one warmer temperature applied at least in part by the thermal therapy-generating element and at least one colder temperature applied at least in part by the cryotherapy-generating element.

In one aspect, the present disclosure relates to methods for supplying temperature modulation therapy to a tissue using a temperature modulation probe. The method may include identifying a modulation pattern, monitoring temperature(s) of the target tissue, and, where needed, adjusting the modulation pattern in real time to effect a desired temperature or temperature profile goal. The method may include continuously supplying therapy to a tissue while automatically adjusting a probe position in a rotational and/or linear direction.

In one aspect, the present disclosure relates to focal laser probes including a shortened lens region for focusing the laser and reducing manufacturing costs. A focal laser probe may be designed, for example, by exposing only a forward directed tip of the laser fiber and shortening the capsule portion of the respective probe to avoid stray energy transmission, for example due to internal reflections. Focal laser probes may be used for providing focal thermal therapy through at least one of ablation, coagulation, cavitation, vaporization, necrosis, carbonization, and reversible thermal cellular damage. The focal emission supplied by focal laser probes provides precision to protect surrounding tissues during thermal therapy, while resulting in minimal unintended tissue changes or damage (e.g. edema) which encourages immediate therapeutic benefit.

In one aspect, the present disclosure relates to cryogenic therapy probes configured for interstitial cryoablation of a tissue. Cryogenic therapy probes can include internal thermal monitoring and real time adjustment of pressure, flow, and/or temperature delivery parameters for adjusting an emission temperature and/or emission pattern. Cryogenic therapy probes may employ Joule-Thomson cooling. An aperture of a fluid delivery tube may be designed for different directional deployment depending upon a desired use for a particular cryogenic therapy probe, such as a side-firing cryogenic therapy probe or a focal cryogenic therapy probe. In some embodiments, an adjustable aperture may be mechanically or electrically adjusted to control flow rate, pressure, and/or deployment patterns (e.g., ranging from focal to diffuse).

In one aspect, the present disclosure relates to probes, probe sheaths, and probe sleeves incorporating one or more recording elements. A recording element may include an electrocardiography (ECG) wire and/or an electroencephalography (EEG) wire. A recording element may be used for lesion localization and assessment at the time of cryotherapy, thermal therapy, or temperature modulation therapy. A recording element may be used to provide positioning and monitoring during functional neurosurgery. In the example of epileptic symptoms, the recording element may be used to confirm positioning of therapeutic energy for treatment of seizure activity. A recording element may be used to confirm disruption of the blood-brain barrier. A recording element may be used for monitoring biorhythms while performing an operation or other therapy. A recording element may be used to apply local tissue stimulation responsive to detection of an abnormal event to regulate cellular behaviors during treatment. A recording element may be used to effect deep brain stimulation during a neurosurgical operation.

In one aspect, the present disclosure relates to recording instruments incorporating one or more recording elements for monitoring internal electrical signals and identifying abnormalities. The recording instruments, for example, may be designed for in vivo deployment for hours or days while monitoring and analyzing signals such as deep brain signals. For signal analysis, leads disposed between recording element contact surfaces and along a shaft of the recording instrument may deliver recorded signals from the contact surfaces to a controller external to the patient for analysis. In one example, a recording instrument includes a cooling tube for delivery of cooling gas or fluid to a cooling zone region of the recording instrument. Further to this example, a temperature sensor such as a thermocouple may be disposed within or adjacent to the cooling zone region of the recording instrument for monitoring a temperature of the recording instrument and/or tissue proximate to the recording instrument. The temperature sensor, in a particular example, may provide temperature data to a thermal readout external to the patient. In an additional particular example, the temperature sensor may provide temperature data to a controller external to the patient for controlling cooling gas or fluid delivery to the cooling zone region.

In one aspect, the present disclosure relates to reduced profile probe designs. Reducing the profile of a probe is desirable for achieving minimally invasive surgery, performing surgical operations upon small bodies such as fetuses, infants, juveniles and animals, and reaching otherwise difficult-to-reach in situ locations without negatively impacting surrounding tissues. A reduced profile probe, for example, can allow entry into small and narrow spaces in the brain while reducing patient injury. A low profile probe may include multiple internal lumens. Low profile probes may be configured with selected materials, lumen structures, and layer structures to provide desired and/or selected mechanical properties including straightness, rigidity, torque strength, column strength, tensile strength, kink resistance, and thermal properties such as thermal stability and thermal stress capacity. Low profile probe dimensions may vary, in some examples, based upon the style of the low profile probe (e.g., thermal therapy, cryotherapy, temperature modulation therapy), the anticipated probe deployment (e.g., intracranial, spinal, cardiac, etc.), the required thermal tolerances of the low profile probe, and/or the required structural tolerances of the probe (e.g., flexible vs. rigid). The following are examples of low profile probe dimensions: the inner shaft of a low profile probe may have an outer diameter of 2.0 mm, within a tolerance of 0.03 mm and an inner diameter of 1.5 mm, within a tolerance of 0.03 mm; the outer shaft may have an outer diameter of 2.25 mm, within a tolerance of 0.03 mm and an inner diameter of 2.07 mm, within a tolerance of 0.03 mm; the shaft may have an outer diameter of approximately 2.1 mm, approximately 2.2 mm, or less than approximately 3.2 mm. In additional examples, the shaft of various low profile probe designs may have an outer diameter of approximately 1.0 mm, 1.2 mm, 1.5 mm, 1.7 mm or 1.8 mm.

With any of the apparatus described within, it may be understood that materials used for manufacture may be selected, in some embodiments, for compatibility with thermal imaging systems, such as Magnetic Resonance Imaging. Thermal imaging-compatible materials, in some examples, may include polymeric material such as nylon, ethylene-tetrafluoroethylene, polyamines, polyimides, and other plastics, quartz, sapphire, crystal structures, and/or glass-type structures. Additionally, small amounts of thermal-imaging tolerant (non-ferromagnetic) metal materials such as titanium and titanium alloys may be included, for example in various connectors for stabilizing positioning of neurosurgical instruments relative to introduction equipment. In further embodiments, material selection may be based in part upon compatibility with various imaging or neurosurgical treatment modalities such as, in some examples, radiofrequency (RF), high-intensity focused ultrasound (HIFU), microwave, and/or cryogenic energy.

In some embodiments, the present disclosure describes a system for deploying at least one signal recording electrode proximate a tissue during interstitial therapy to the tissue, the system including: an interstitial therapy instrument including a tip region and a shaft region, where the interstitial therapy instrument includes at least one therapy generating element, where the at least one therapy generating element includes one of a) a thermal therapy-generating element for thermal therapy emission via the tip region and b) a cryotherapy-generating element for cryogenic therapy emission via the tip region; and at least one signal recording element configured for deployment proximate to the tip region, where the at least one signal recording element includes one of an electrocardiography recording element, an electroencephalography recording element, and a stereo electroencephalography recording element.

In some embodiments, the system includes a controller including processing circuitry and a memory having instructions stored thereon, where the instructions, when executed by the processing circuitry, cause the processing circuitry to, while the interstitial therapy instrument is positioned proximate a tissue, receive signal recordings from the at least one signal recording element, analyze the signal recordings to identify an abnormal signal pattern, and responsive to identifying the abnormal signal pattern, cause at least one of i) adjustment of an emissive output of a first therapy generating element of the at least one therapy generating element, ii) adjustment of a therapeutic profile for delivering therapy to the tissue via the interstitial therapy instrument, iii) adjustment of a linear position of the interstitial probe, iv) adjustment of a rotational position of the interstitial therapy instrument, and v) output of at least one of visual information and audible information regarding the abnormal signal pattern for the attention of an operator. The instructions may cause the processing circuitry to, prior to receiving the signal recordings, cause extension of at least a first signal recording element of the at least one signal recording element along the tip region of the interstitial therapy instrument. The first signal recording element may be extended from the shaft portion of the interstitial therapy instrument. The instructions may cause the processing circuitry to, after receiving the signal recordings and prior to adjusting the emissive output of the first therapy generating element, cause retraction of the first signal recording element such that the first signal recording element will not interfere with the first therapy generating element. Identifying the abnormal signal pattern may include identifying a signal pattern associated with a lesion. The instructions may cause the processing circuitry to, before receiving the signal recordings, cause emission of the cryotherapy-generating element directed to the tissue; receive initial signal recordings from the at least one signal recording element, analyze the initial signal recordings to identify a hibernation pattern; and cause cessation of emission of the cryotherapy-generating element; where the signal recordings are captured while the tissue is warming.

In some embodiments, the system includes a flexible sleeve, where the flexible sleeve surrounds the interstitial therapy instrument and includes the at least one signal recording element. The system may include a guide sheath including a number of lumens, where the interstitial probe is disposed within a first lumen of the number of lumens and a first signal recording element of the at least one signal recording element is disposed within a second lumen of the number of lumens.

In some embodiments, the at least one signal recording element includes at least three signal recording elements. The at least three signal recording elements may be provided as rings surrounding a circumference of the interstitial therapy instrument. The rings may be formed along the shaft region of the interstitial therapy instrument.

In some embodiments, the present disclosure describes a focal laser induced interstitial thermal therapy probe for treatment of a tissue, including: a transparent lens capsule; a laser fiber including a sheath portion and an exposed tip, where the exposed tip is disposed within the transparent lens capsule; a shaft portion fixed to the transparent lens capsule, where the laser fiber extends along the shaft portion; and a cooling supply tube disposed within the shaft portion for delivering at least one of a cooling fluid and a cooling gas to the transparent lens capsule; where the exposed tip is configured to direct focal energy through a tip portion of the transparent lens capsule in a forward direction, and the length of the transparent lens capsule is configured to minimize energy transmissions outside of the forward direction. The tip portion of the transparent lens capsule may be substantially flat in shape. The exposed tip may be substantially flat in shape. In further embodiments, the tip portion of the transparent lens capsule and/or the exposed tip may be rounded, torpedo-shaped, or pointed.

In some embodiments, the present disclosure describes a system for providing interstitial cryotherapy to a tissue, the system including: an interstitial cryogenic probe, including a shaft region and a tip region, where a distal end of the tip region is affixed to a proximal end of the shaft region. The system may include an injection tube disposed within the shaft region for delivering a refrigerant to the tip region, and a temperature sensor disposed within the shaft region. The system may include a controller including processing circuitry and a non-transitory computer readable medium having instructions stored thereon for controlling emission of the interstitial cryogenic probe, where the instructions, when executed by the processing circuitry, cause the processing circuitry to, during cryotherapy of the tissue, receive temperature signals from the temperature sensor, and responsive to the temperature signals, adjust at least one of a pressure, a flow rate, and a temperature of refrigerant delivered to the injection tube. The temperature sensor may be a thermocouple or fiber optic thermometer. The injection tube and tip region may be configured for Joule-Thomson cooling. The interstitial cryogenic probe may include a vacuum return lumen to direct evaporated refrigerant towards the shaft region of the interstitial cryogenic probe. An orifice of the injection tube may be configured for side-firing emission of refrigerant. The interstitial cryogenic probe may include a porous plug at an orifice of the injection tube. Adjusting the flow rate may include modulating the flow of refrigerant in an on-off pattern.

In some embodiments, the present disclosure describes an interstitial probe for performing temperature modulation therapy to a tissue, the interstitial probe including: a shaft region; a tip region; at least one thermal therapy-generating element for thermal therapy emission via the tip region; at least one cryotherapy-generating element for cryogenic therapy emission via the tip region; processing circuitry disposed within the shaft region; and a memory disposed within the shaft region, the memory having instructions stored thereon for causing emission of a number of thermal modulation patterns, where each thermal modulation pattern of the number of thermal modulation patterns includes at least one higher thermal output corresponding to activation of a first thermal therapy element of the at least the thermal therapy-generating element for a first time interval, and at least one lower thermal output corresponding to activation of a first cryogenic therapy element of the at least one cryogenic therapy element for a second time interval different than the first time interval. The instructions, when executed by the processing circuitry, may cause the processing circuitry to receive selection of a first thermal modulation pattern of the number of thermal modulation patterns, and activate temperature modulation therapy utilizing the modulation pattern. The memory may include a programmable memory element, the interstitial probe further including at least one communication connection for programming the programmable memory element with one or more additional thermal modulation patterns.

In some embodiments, the present disclosure describes a low profile interstitial probe for effecting at least one of thermal therapy and cryotherapy to a tissue, the low profile interstitial probe including a shaft, including at least one outer layer and at least one inner layer, where an outermost layer of the at least one outer layer has a first set of mechanical properties including at least two of the following: straightness, rigidity, torque strength, column strength, tensile strength, kink resistance, thermal stability, and thermal stress capacity, and an approximate outer diameter of less than 3.3 mm, and an innermost layer of at least one inner layer has a second set of mechanical properties including at least two of the following: straightness, rigidity, torque strength, column strength, tensile strength, kink resistance, thermal stability, and thermal stress capacity, and an approximate maximum inner diameter of at least 1.5 mm; The low profile interstitial probe may include a transparent lens capsule through which energy is delivered to the tissue during treatment, where a distal end of the transparent lens capsule is connected to a proximal end of the shaft region; and an energy emission element may be disposed at least in part within the transparent lens.

The low profile interstitial probe may include a number of lumens formed within the innermost layer, where the maximum inner diameter corresponds to a widest diameter measurable between two or more adjacent lumens. A first lumen of the number of lumens may be configured to carry an energy emission medium to the energy emission element; and a second lumen of the number of lumens may be configured to deliver cooling gas to the transparent lens capsule.

The outermost layer may be configured to act a protective barrier in case of breakage of a layer of the shaft directly abutting an inner surface of the outermost layer. The outermost layer may be a thin-walled polyether ether ketone (PEEK) plastic; and the outermost layer may partially overlap the transparent lens capsule. The outermost layer may be linearly aligned with the remaining layers of the at least one outer layer and the at least one inner layer to provide a counterbore region, where a distal portion of the transparent lens capsule is permanently affixed to the counterbore region. The transparent lens capsule may be composed of machined sapphire.

The foregoing general description of the illustrative implementations and the following detailed description thereof are merely exemplary aspects of the teachings of this disclosure, and are not restrictive.

### BRIEF DESCRIPTION OF THE DRAWINGS

A more complete appreciation of this disclosure and many of the attendant features thereof will be readily obtained as the same becomes better understood by reference to the following detailed description when considered in connection with the accompanying drawings, wherein:

FIGS. 1A and 1B illustrate components of an example probe;

FIGS. 1C and 1D illustrate components of an example variable length probe apparatus;

FIGS. 1E and 1F illustrate an example depth locking element for use with the variable length probe of FIGS. 1C and 1D;

FIG. 1G illustrates an example cut-away view of a flexible umbilical portion of the variable length probe of FIGS. 1C and 1D;

FIG. 1H illustrates an example transition element positioned between the variable length probe and the flexible umbilical of the variable length probe apparatus of FIGS. 1C and 1D;

FIG. 2A illustrates an example probe driver;

FIGS. 2B-2D illustrate an example probe follower for use with the probe driver of FIG. 2A;

FIGS. 2E-2G illustrate an example probe follower with a low profile design;

FIG. 3A illustrates an example probe design with built-in thermal modulation;

FIG. 3B illustrates an example focal laser fiber;

FIG. 4 illustrates an example probe configured for focal cryotherapy;

FIGS. 5A through 5E illustrate example options for incorporation of a signal recording element with a thermal therapy, cryotherapy, or temperature modulation therapy probe such as the probe of FIG. 3A or the probe of FIG. 4;

FIGS. 5F through 5H illustrate example options for designing a recording instrument;

FIG. 5I illustrates a flow chart of a method for using interstitial signal recording elements, such as the recording elements described in relation to FIGS. 5A through 5G.

FIG. 6A illustrates a graph of an example modulation pattern for temperature modulation therapy;

FIG. 6B is a diagram of an example effect upon a tissue caused by temperature modulation therapy by a temperature modulation probe;

FIG. 7A is a graph of example temperature ranges for causing various effects on tissue via thermal therapy;

FIG. 7B is a graph of example temperature ranges for causing various effects on tissue via cryotherapy;

FIG. 8 is a flow chart of an example process for effecting a temperature modulation therapy using a temperature modulation probe;

FIG. 9 is a longitudinal cross-sectional view through an alternative form of a probe that provides a flow of cooling fluid to the end of the probe for cooling the surrounding tissue;

FIG. 10 is a cross-sectional view along the lines 10-10 of FIG. 9;

FIG. 11 is a longitudinal cross-sectional view through a further alternative form of probe which provides a flow of cooling fluid to the end of the probe for cooling the surrounding tissue;

FIG. 12 is a cross-sectional view along the lines 12-12 of FIG. 11;

FIGS. 13A and 13B illustrate a first example multi-lumen low profile probe design;

FIGS. 14A and 14B illustrate a second example multi-lumen low profile probe design;

FIGS. 15A through 15C illustrate an example low profile probe design with a multi-layer shaft;

FIGS. 16A through 16H illustrate alternative depth locking element configurations for use with the variable length probe of FIGS. 1C and 1D; and

FIG. 17 is a block diagram of an example computing system hardware design.

### DETAILED DESCRIPTION OF THE ILLUSTRATIVE EMBODIMENTS

In the drawings, like reference numerals designate identical or corresponding parts throughout the several views. Further, as used herein, the words “a,” “an” and the like generally carry a meaning of “one or more,” unless stated otherwise.

Further, in individual drawings figures, the components/features shown are drawn to scale to exemplify a particular implementation. For some drawings, components/features are drawn to scale across separate drawing figures. However, for other drawings, components/features are shown magnified with respect to one or more other drawings. Measurements and ranges described herein relate to exemplary implementations and can identify a value or values within a range of 1%, 2%, 3%, 4%, 5%, or, preferably, 1.5% of the specified value(s) in some implementations.

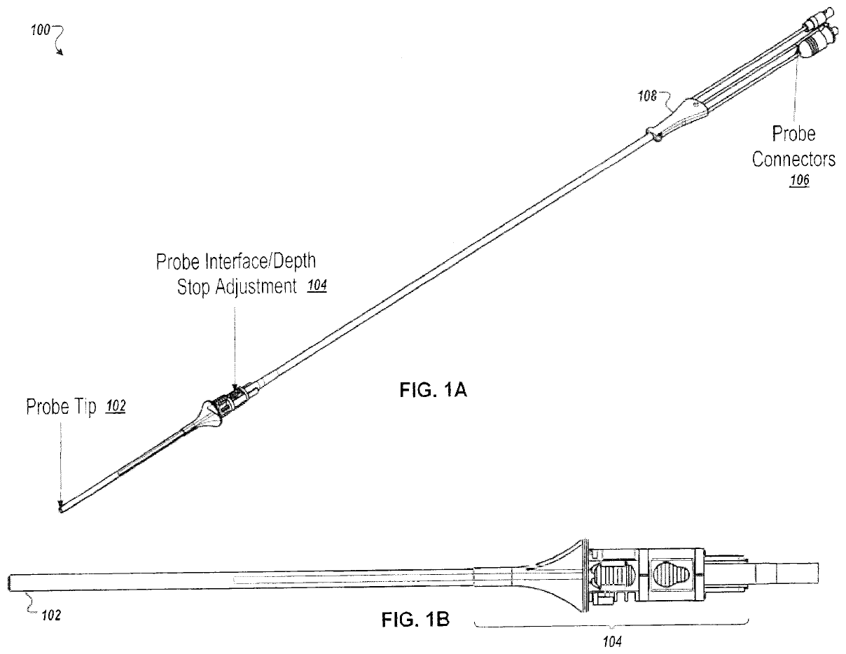

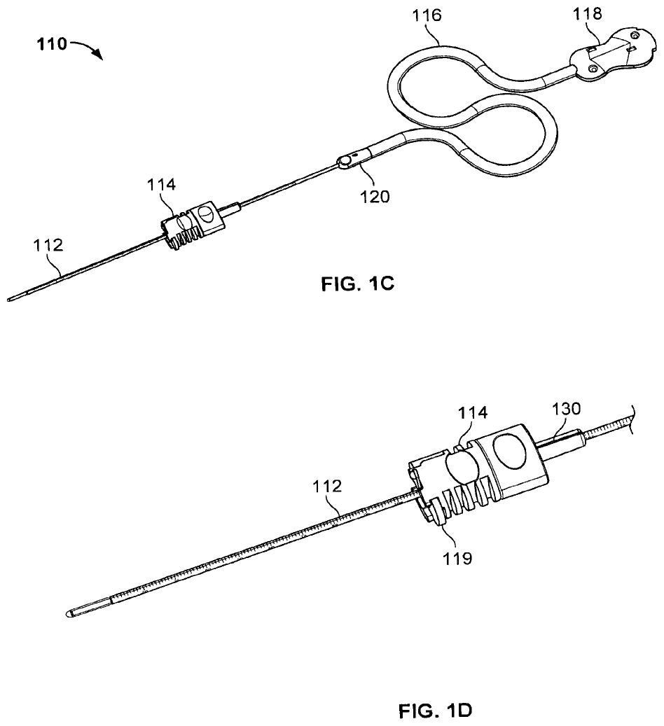

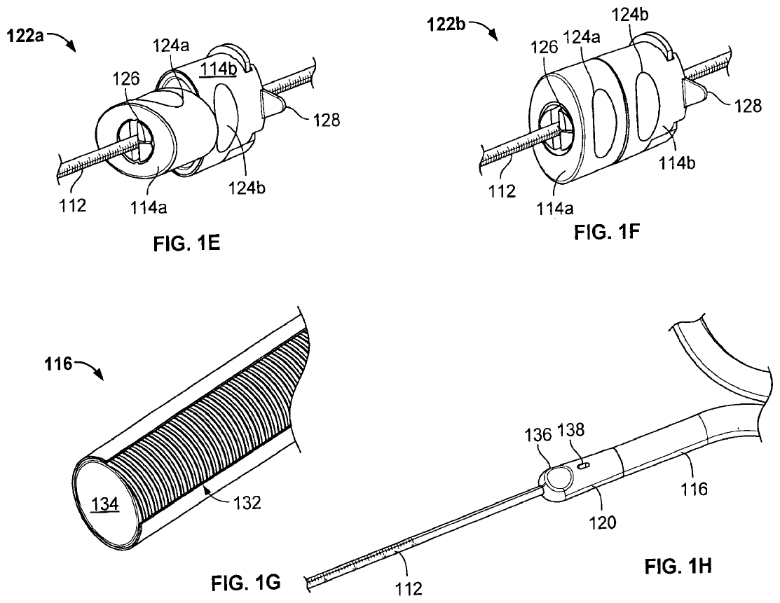

FIGS. 1A and 1B illustrate exemplary aspects of a probe. Types of probes that can be utilized with the components and procedures discussed herein include laser, radiofrequency (RF), high-intensity focused ultrasound (HIFU), microwave, cryogenic, chemical release, which may include photodynamic therapy (PDT), and drug releasing probes.

Further probes can include temperature modulation probes including at least one thermal therapy-generating element (e.g., RF, HIFU, microwave, laser, electrical heat, heating fluid or supercritical fluid, heating gas, etc.) and at least one cryotherapy-generating element (e.g., cooling gas, cooling fluid, etc.). Each of the at least one thermal therapy-generating element and the at least one cryotherapy-generating element may be configured to emit respective thermal or cryo energy in a side-firing, focal, or diffuse manner. In a particular example, a temperature modulation probe includes a circumferentially emitting thermal therapy-generating element and a circumferentially emitting cryotherapy-generating element. The temperature modulation probes of the present disclosure may be designed for insertion into a body cavity, insertion into vascular system, or interstitial deployment.

Treatments in accordance with the descriptions provided in this disclosure include treatments that ablate a tissue to destroy, inhibit and/or stop one or more or all biological functions of the tissue. Ablation agents include, but are not limited to, laser, RF, HIFU, microwave, cryogenic, PDT and drug or chemical release. A corresponding probe and/or another instrument, such as a needle, fiber or intravenous line can be utilized to effect treatment by one or more of these ablation agents. Treatments in accordance with the descriptions provided in this disclosure include treatments that create temporary or permanent physical-biological effects to tissue including freezing, freeze-thawing, hyperthermia, coagulation, and/or vaporization of tissues. The temporary or permanent physical-biological effects can include alterations in biological function of organelles, cell constituents including DNA, cells, extracellular matrices, tissues, and/or body fluids. In a particular example, the treatment may cause the DNA, cells, extracellular matrices, tissues, and/or body fluids to be more receptive or sensitive to additional therapies or manipulations such as, in some examples, radiation therapy or chemotherapy. In another example, temporary or permanent physical-biological effects can include detecting and/or treating neurological disorders. Epileptic activity, in particular, may be suppressed or stopped through a hypothermia treatment (e.g., effecting thermal therapy within a range of approximately 16 to 33° C.). In another illustration, Parkinson's symptoms may be similarly suppressed or stopped. In a further example, the treatment may cause hemostasis, reduction or dissolution of thrombi or emboli, alteration of functional membranes including the blood-brain barrier, and/or renal filtration. The physical-biological effects may be caused directly by temperature change to the DNA, cells, extracellular matrices, tissues, and/or body fluids or indirectly (e.g., downstream) from the temperature change, such as alterations in heat shock proteins or immune system or cell reaction or status.

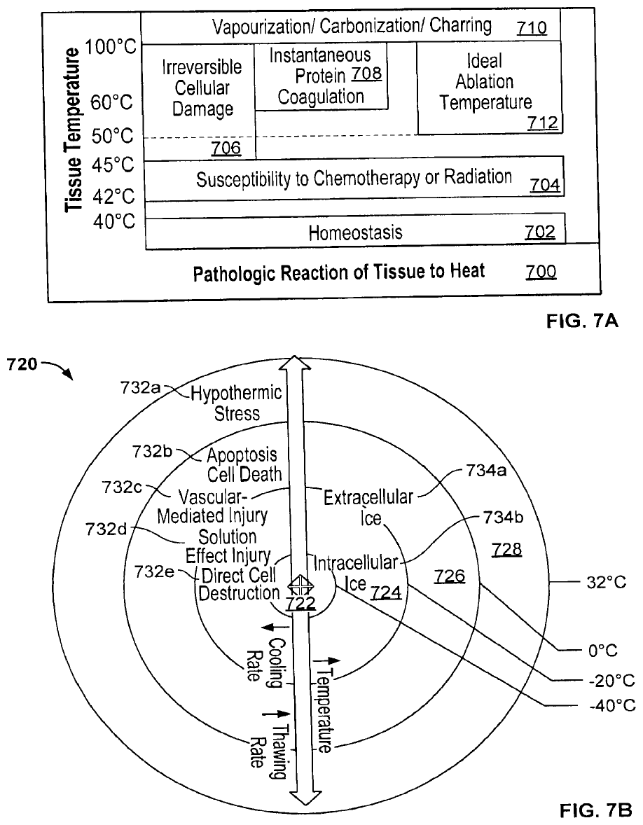

The alterations, in some circumstances, may have longer range effects. For example, the treatment may cause the cells, tissues, and/or body fluids to be more receptive or sensitive to additional therapies or manipulations for an extended time period. The extended time period, in some examples, may include a day, a number of days, a week, a number of weeks, and/or a number of months. In this manner, the physical-biological effects triggered by the treatment may provide the opportunity for ongoing therapy throughout the extended time period. In a particular example, application of therapy triggering physical or biological effects may be followed up, within a matter of hours, days, weeks, or even months, with supplemental therapy such as intravenous drug therapy, chemotherapy, and/or radiation therapy or other therapies that benefits from the increased sensitivity produced by the physical or biological effects. Turning to FIG. 7A, a graph 700 illustrates example temperature ranges for causing various effects on tissue via thermal therapy, such as homeostasis 702 (up to 40° C.), susceptibility to chemotherapy or radiation 704 (about 42° C. to 45° C.), irreversible cellular damage 706 (about 45° C. to 100° C.), instantaneous protein coagulation 708 (about 55° C. to 100° C.), and vaporization/carbonization/charring 710 (above 100 C ° C.). The homeostasis 702 effect, for example, may be caused at temperatures below those traditionally labeled as “hyperthermia” treatment. Hyperthermia treatment, effected for example within an approximate range of 41-45° C., can be used to effect reversible physical and/or biological changes. These changes, as indicated in FIG. 7A, can increase sensitivity or receptiveness to additional therapies, such as chemotherapy or radiation. Thermal ablation therapies, such as those applied at temperatures exceeding 50° C., cause irreversible cellular damage, including instantaneous protein coagulation 708, vaporization, carbonization, or charring 710.

The ranges illustrated in FIG. 7A represent estimated bands of opportunity for causing the various effects 702-710 upon tissue, while more precise temperature ranges depend upon a number of factors such as tissue type, tissue location, baseline temperature. Further, the graph 700 illustrates an “ideal” ablation temperature range 712 of approximately 50° C. to 100° C. The ideal ablation temperature range 712, starting about 5 degrees hotter than irreversible cellular damage range 706, may be representative of a range in which there is desired degree of confidence that therapy will cause total cellular death without unwanted tissue damage (vaporization/carbonization/charring 710).

Turning to FIG. 7B, a graph 720 illustrates example temperature ranges for causing various effects on tissue via cryotherapy. For example, within a first thermal band 722 (up to −40° C.) as well as a second thermal band 724 (−40° C. to −20° C.), direct cell destruction 732e (e.g., irreversible cellular damage) may occur during cooling 730a, while intercellular ice 734b may develop. Also within the second thermal band 724, solution effect injury 732d may occur and extracellular ice 734a may develop. The solution effect injury 732d and extracellular ice 734a carries over into a third thermal band 726 (between −20° C. and 0° C.). The second thermal band 724 and third thermal band 726 identify thermal regions of treatment where application of cooling over time can cause irreversible cellular damage. Also shared between the second thermal band 724 and the third thermal band 726, vascular-mediated injury 732c may occur. Within the third thermal band 726, apoptosis cell death 732b may occur. Finally, within a fourth thermal band 728 (0° C. to 32° C.), hypothermic stress may result. The fourth thermal band 728 provides opportunity for treatments involving reversible cellular damage, such as identifying and/or suppressing neurological disorder symptoms (e.g., epilepsy) or increasing sensitivity or receptiveness to additional therapies, such as IV drug therapy, chemotherapy or radiation, including changes to cellular membranes to allow for increased (or decreased) absorption, transmission, or other movement of a chemical, drug, biological agent, or cell. An energy output pattern of a temperature modulation probe includes a modulated output pattern, varying between at least one warmer temperature applied at least in part by one or more thermal therapy-generating elements and at least one cooler temperature applied at least in part by one or more cryotherapy-generating elements. In certain embodiments, a particular energy output pattern may be developed based upon the type of thermal therapy-generating elements and cryotherapy-generating element included within the probe, an emission style of the probe tip (e.g., side-firing, focal tip, diffuse tip, etc.), and/or the depth of the region of interest or the targeted tissue area (e.g., based in part on the shape of a tumor region, etc.).

An energy output pattern of a probe, such as a laser probe or HIFU probe, in certain embodiments, includes a pulsed output pattern. For example, a higher power density may be achieved without causing tissue scorching by pulsing a high power laser treatment for x seconds with y seconds break between (e.g., allowing for tissue in the immediate vicinity to cool down by only activating the cryotherapy-generating element). In a particular example, the energy output pattern of a probe may include a ten Watt output of the thermal therapy-generating element for two seconds while maintaining activation of the cryotherapy-generating element, followed by a one second period of inactivity of the thermal therapy-generating element while maintaining activation of the cryotherapy-generating element. Conversely, the thermal therapy-producing element may remain activated, in some pulsed output patterns, while modulating activation of the cryotherapy-generating element.

An energy output pattern of a probe, in certain embodiments, includes a refined temperature modulated pattern, where both the thermal therapy-generating element(s) and the cryotherapy-generating element(s) is/are constantly activated, while power levels, emission levels, flow rates, and/or energy levels are varied between the elements to cycle between cooler and warmer output.

In certain embodiments, a particular energy output pattern may be developed based upon the type of probe (e.g., laser, HIFU, etc.), an emission style of the probe tip (e.g., side-firing, diffuse tip, etc.), and/or the depth of the ROI or the targeted tissue area (e.g., based in part on the shape of a tumor region, etc.).

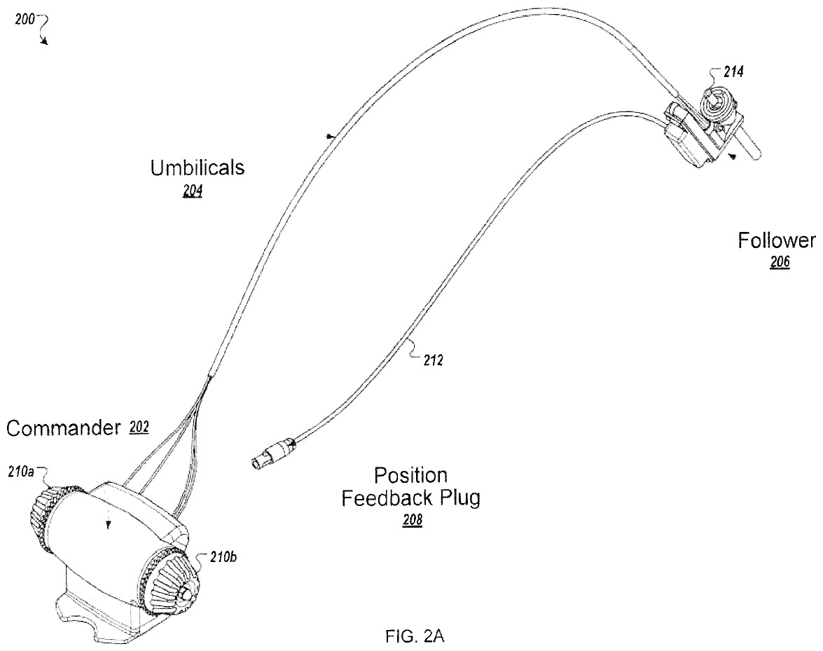

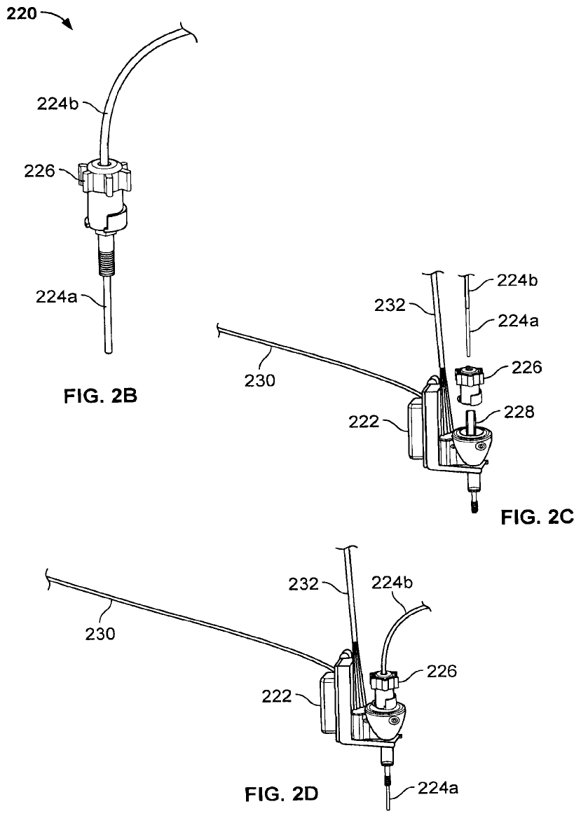

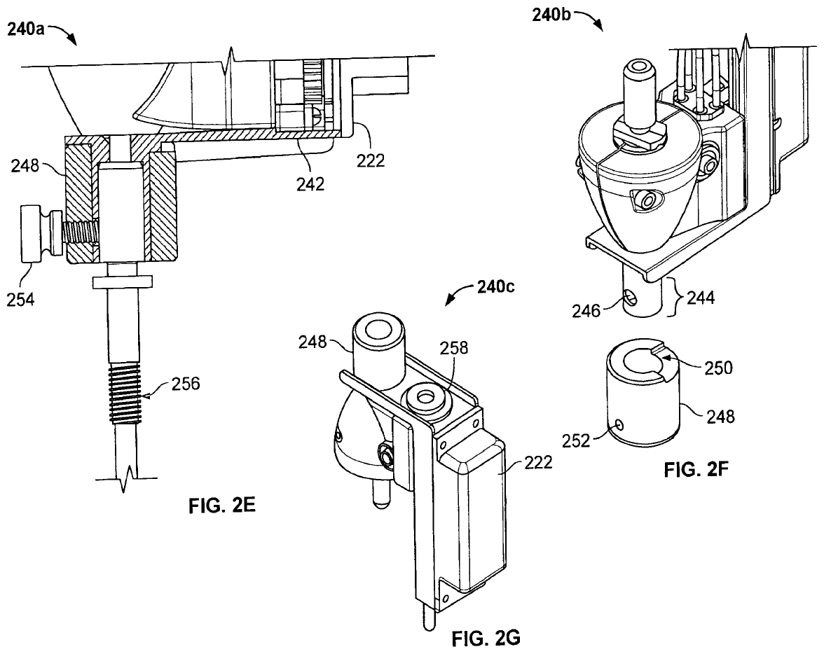

In certain embodiments, a treatment pattern includes effecting treatment while concurrently or simultaneously moving the probe (e.g., linearly and/or rotationally). For example, a thermal therapy, cryotherapy, or temperature modulation probe may be automatically rotated (e.g., using a commander and follower as described in relation to FIG. 2A) while an emission pattern and/or modulation pattern is simultaneously or concurrently adjusted to effect treatment to a desired depth based upon a particular geometry of the region of interest.

An example probe apparatus 100 is shown in FIG. 1A and FIG. 1B. A probe tip 102 indicates an insertion end of the probe apparatus 100. A probe interface/depth stop adjustment 104 provides an interface for cabling, as well as for alignment with the probe driver and/or the stereotactic miniframe. An end opposite the insertion end includes probe connectors 106 for energy delivery, cooling, etc. extending from a probe interface boot 108. FIG. 1B is an enlarged view of the probe interface 104 to probe tip 102 portion of the probe apparatus 100 shown in FIG. 1A.

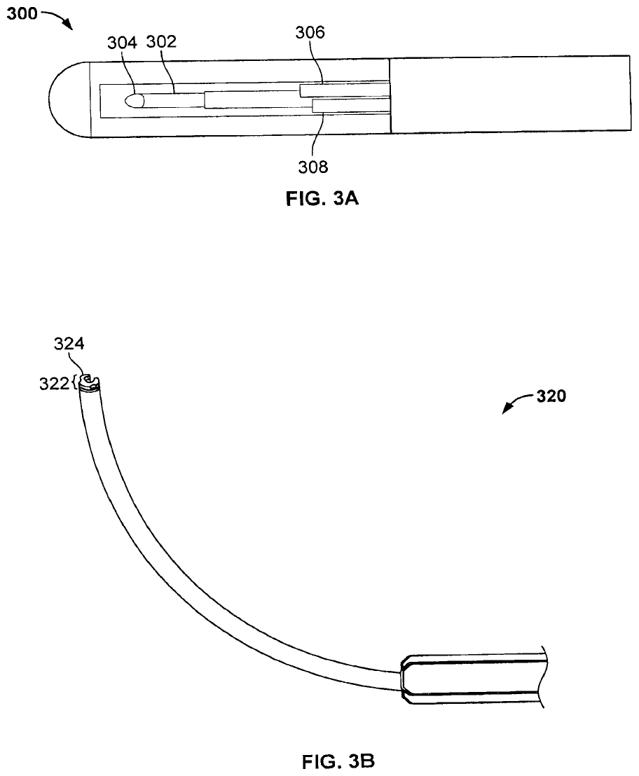

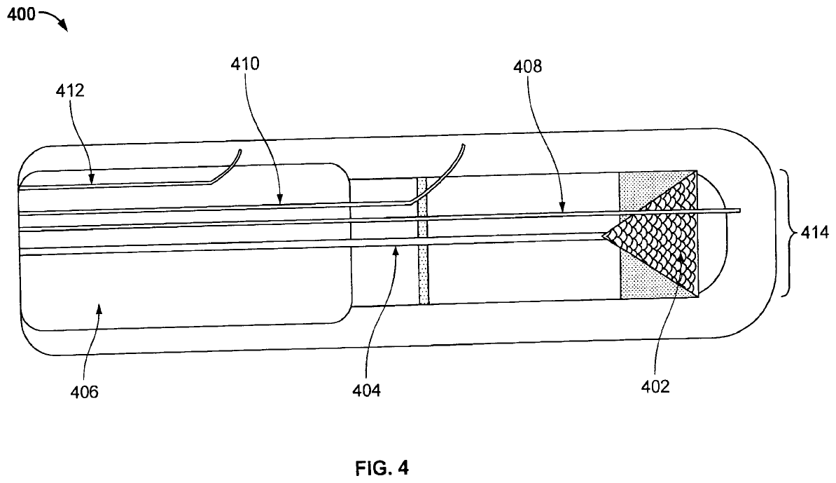

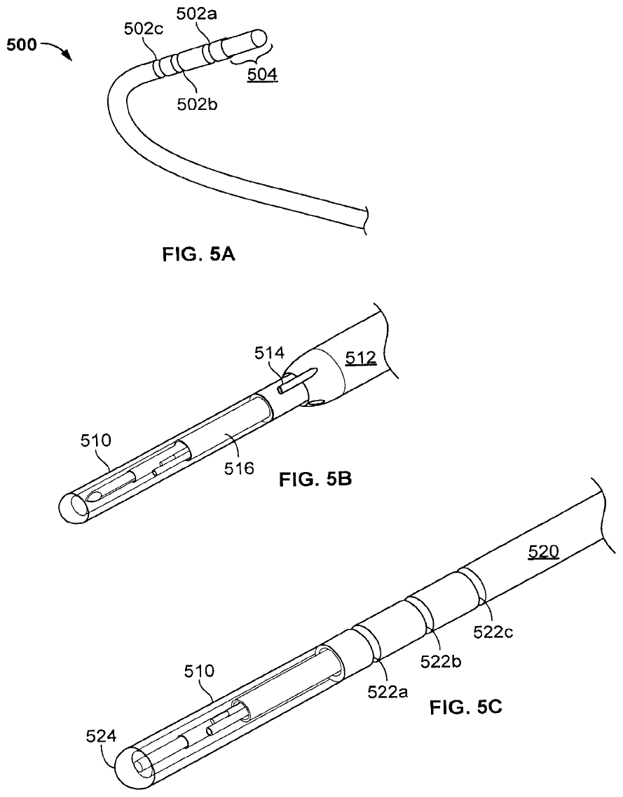

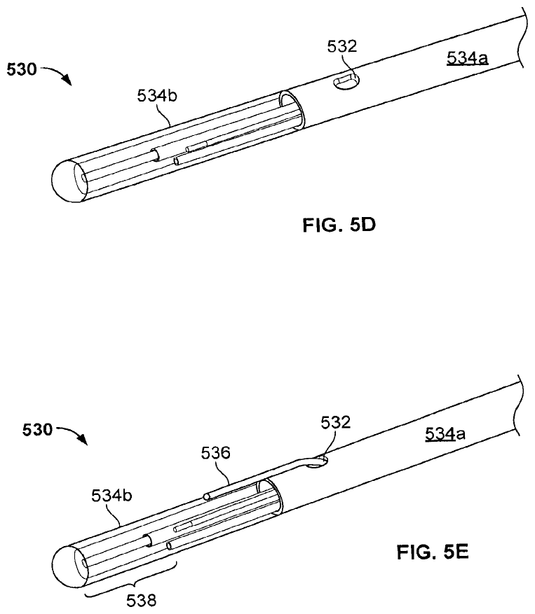

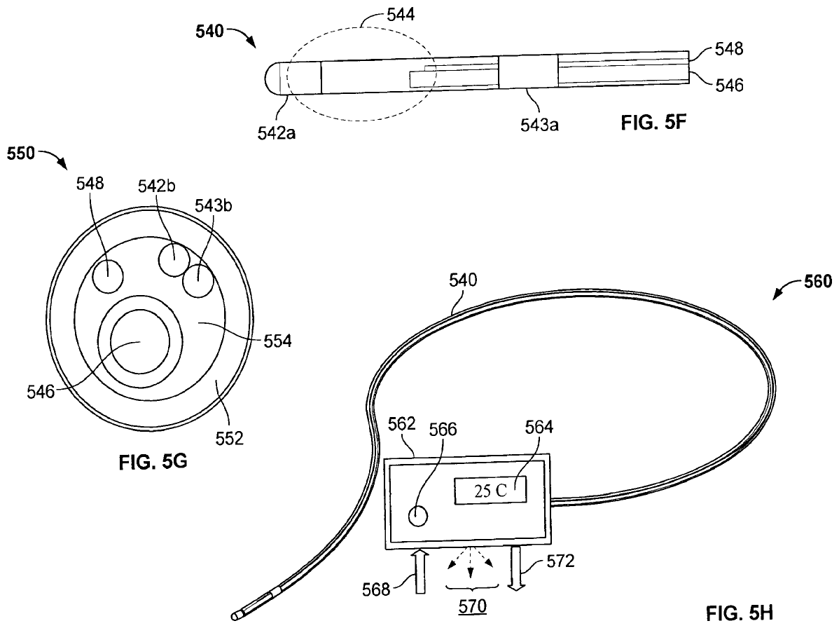

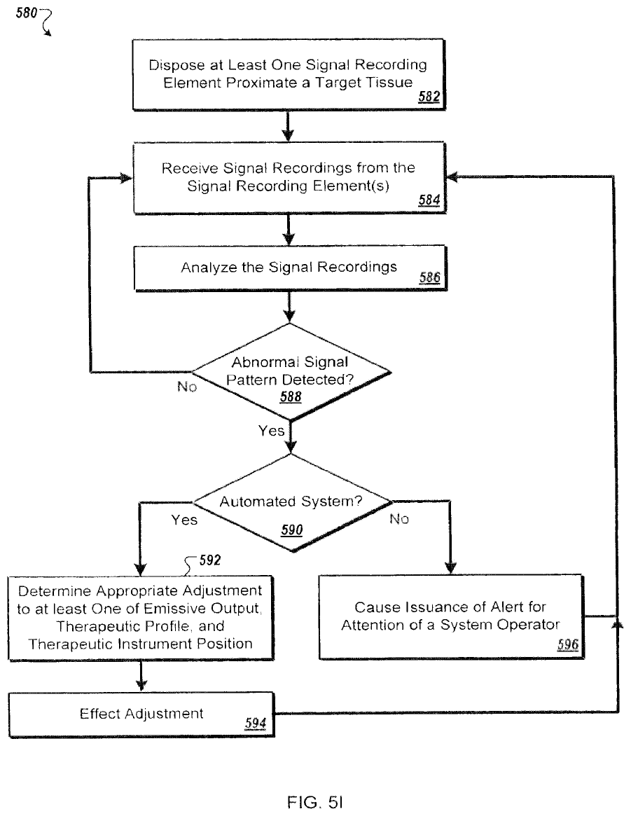

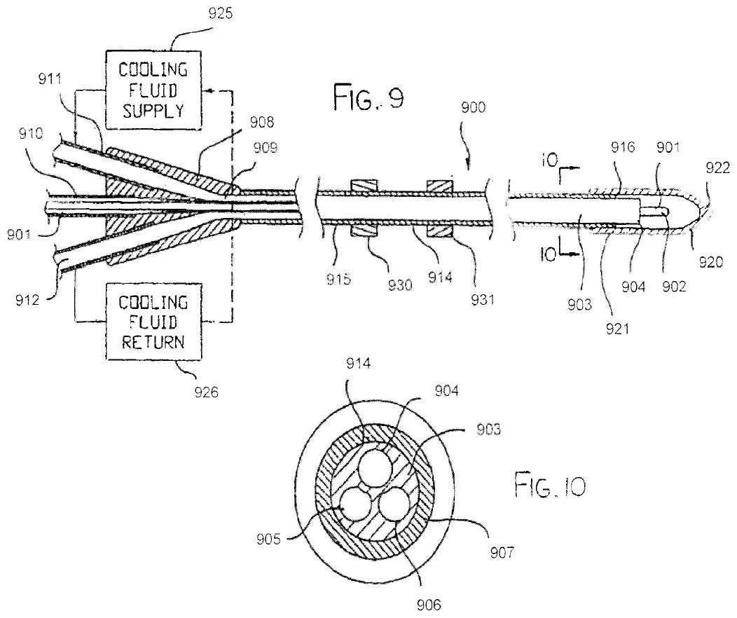

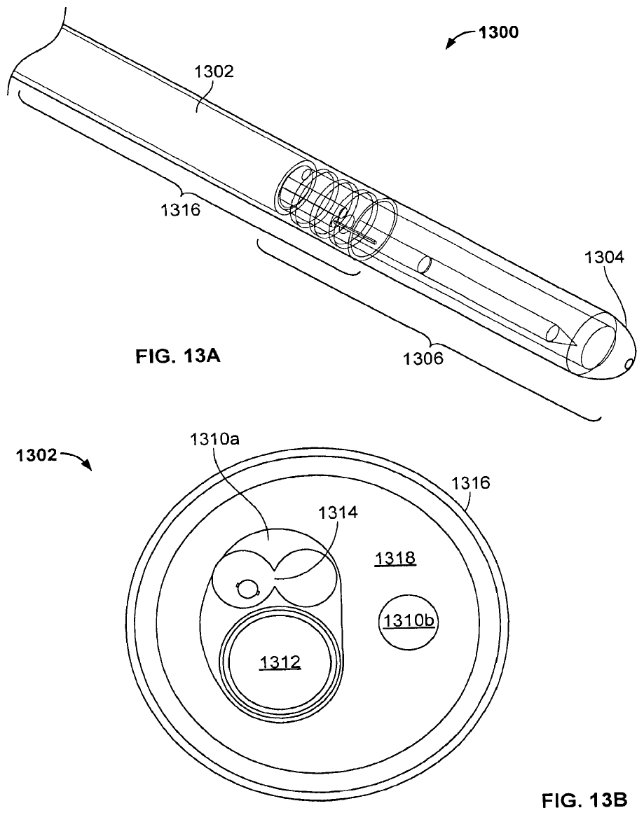

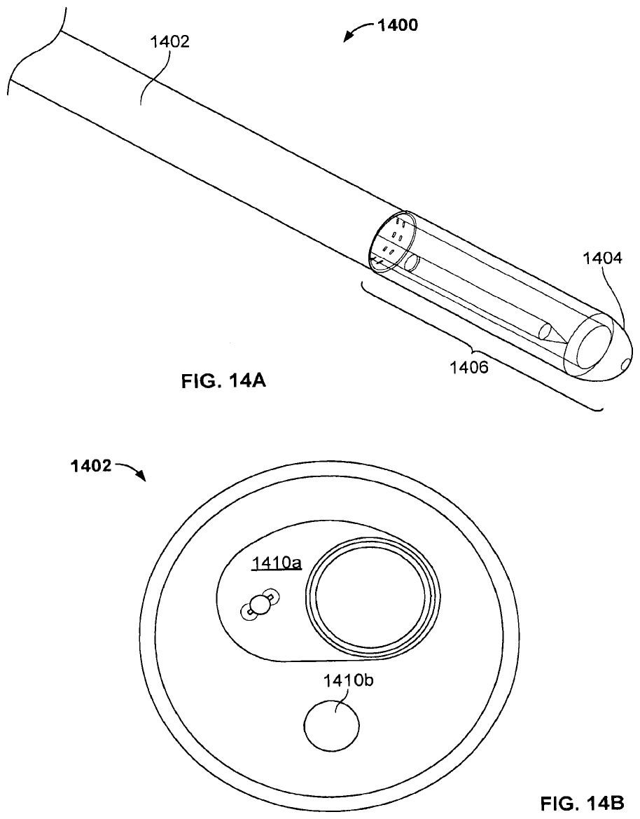

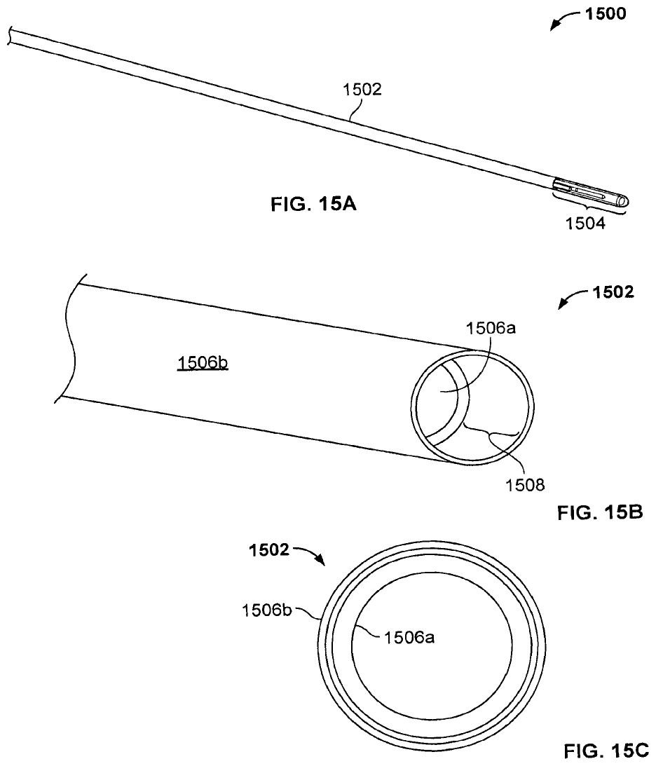

In an example embodiment of probe apparatus as discussed in relation to FIG. 1A and FIG. 1B, turning to FIG. 1C, a variable length probe apparatus 110 has a variable length probe structure including a probe 112 and an adjustable depth stop 114 to facilitate access to both shallow and deep targeted tissue areas; that is, the same probe 112 can accommodate lesions located at varying depths by repositioning over the adjustable depth stop 114. Beyond the probe 112 is a flexible umbilical sheath 116 for carrying inputs and outputs (e.g., energy, control signals, cooling gas or fluid, and/or heating gas or fluid) between the probe 112 and a control unit (not illustrated). At the junction of the probe 112 and the flexible umbilical sheath 116 is a transitional part 120 configured for ease of grasping and manipulation of the probe 112 when positioning the probe 112 in a target tissue region. The general design of the variable length probe apparatus 110 shown in FIG. 1C contains elements shown in FIG. 1A and FIG. 1B, such as a probe interface boot 118 which would lead to the probe connectors 106 illustrated in FIG. 1A. Further, the probe 112 may be designed to achieve the desired and/or selected thermal and/or mechanical properties discussed throughout in relation to the various probe designs, such as the probe apparatus 110 of FIG. 1C, the probe apparatus 224 of FIG. 2B, the probe 300 of FIG. 3A, the probe 320 of FIG. 3B, the probe 400 of FIG. 4, the probe 500 of FIG. 5, the probe 510 of FIGS. 5B and 5C, the probe 530 of FIGS. 5D and 5E, the probe 900 of FIG. 9, the probe of FIG. 11, the probe 1300 of FIG. 13A, the probe 1400 of FIG. 14A, and/or the probe 1500 of FIG. 15A. In some embodiments, the variable length probe apparatus 110 is capable of remote operation, for example using a probe driver apparatus such as the probe driver discussed in relation to FIG. 2A.

As illustrated in FIG. 1D, an enlarged view of the probe 112 and the adjustable depth stop 114 demonstrates measured gradations (e.g., centimeters, millimeters, inches, etc.) printed along the shaft of the probe 112. An operator may align the measured gradations with the adjustable depth stop 114, for example, by sliding a position of the adjustable depth stop 114 along the shaft of the probe 112 until the desired depth measurement aligns with the probe end of the adjustable depth stop 114. In another example, the operator may align the desired depth measurement with the sheath end of the probe 112 (e.g., for clearer visibility). In this example, the gradations may be applied to the shaft of the probe 112 to compensate for a length of the adjustable depth stop 114. In a further embodiment (not illustrated), the adjustable depth stop 114 may include a window (e.g., cut-out portion, clear portion, or clear, magnified portion) for aligning a desired depth measurement while slidably positioning the adjustable depth stop 114. In this example, the gradations may be applied to the shaft of the probe 112 to compensate for the portion of the length of the adjustable depth stop 114 from the probe end to the depth selection window.

Instead or in addition to the printed gradations, in other embodiments, the shaft of the probe 112 may include a series of mating points for mating with the adjustable depth stop 114. For example, the shaft of the probe 112 may include a series of bumps (e.g., every 5 millimeters, 10 millimeters, etc.) and the adjustable depth stop 114 may include one or more mating depressions for engaging with at least one of the series of bumps. The mating points, for example, may be used to more precisely align the adjustable depth stop 114 with a particular depth setting.

Using the adjustable depth stop 114 with the guidance of the printed gradations upon the shaft of the probe 112, an operator may modify the probe length in situ. For example, during a patient operation, after applying a procedure at a first selected depth, an operator may vary the length or depth relatively rapidly to a second selected depth. To allow for varying the probe length of the probe 112 in-situ while controlling the internal deployment of the probe tip, the shaft of the probe 112 may be configured with a selected kink resistance value, in addition to column strength and/or torque strength and/or tensile strength and/or thermal stability.

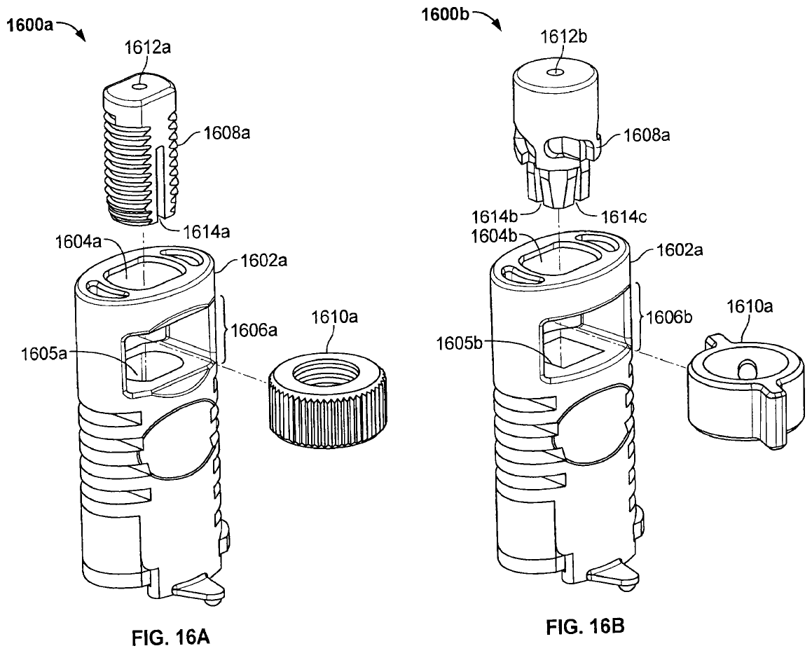

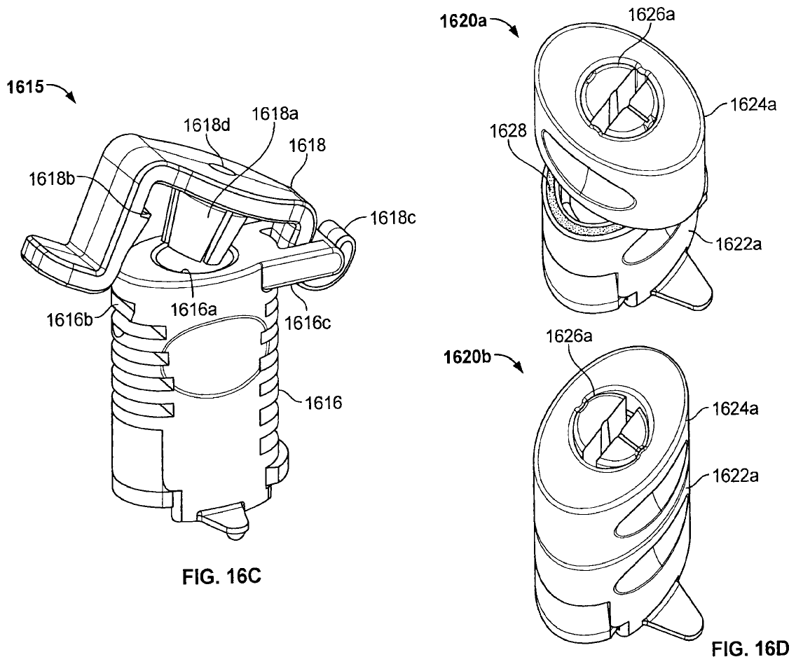

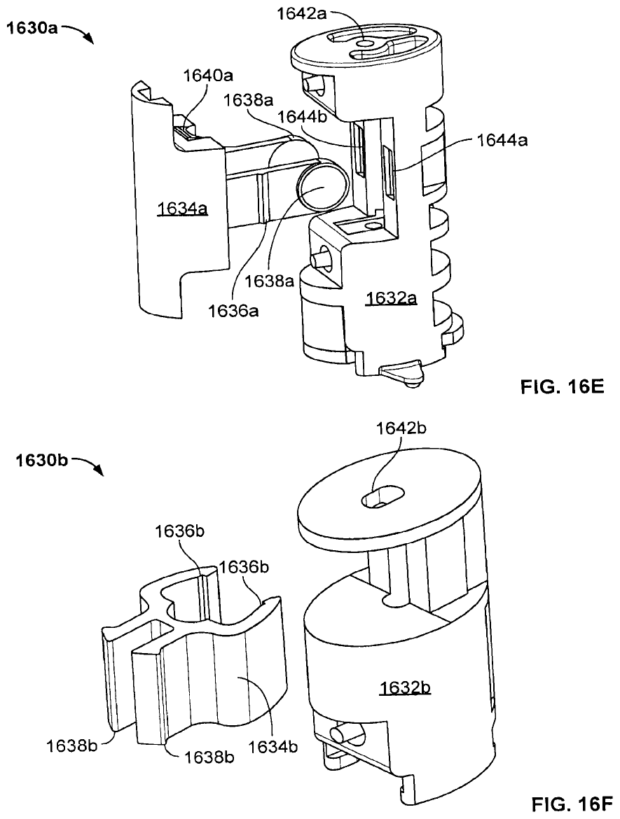

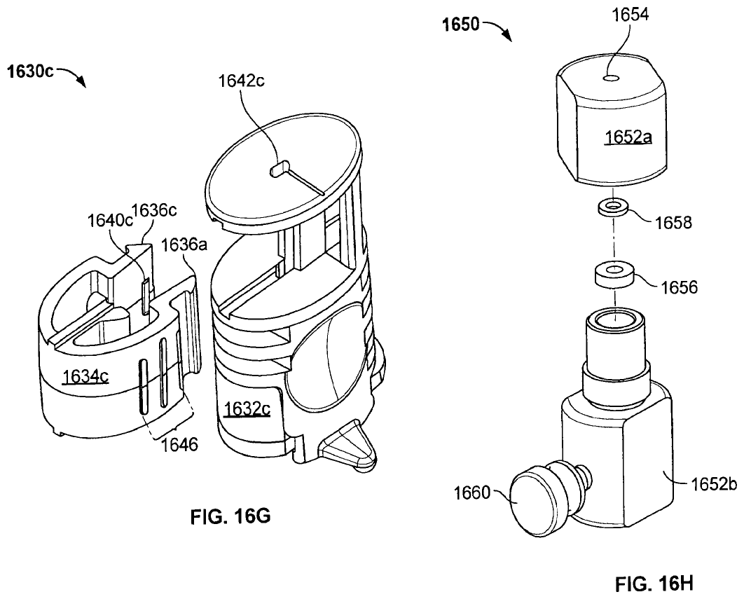

In some embodiments, the probe apparatus 110 is compatible with a low profile trajectory guide (e.g., as described in U.S. patent application Ser. No. 14/661,194 entitled “Image-Guided Therapy of a Tissue” and filed Mar. 18, 2015 and U.S. patent application Ser. No. 14/661,212, entitled “Image-Guided Therapy of a Tissue” and filed Mar. 18, 2015, the contents of each of which is incorporated herein by reference in its entirety) and a minibolt (as described in U.S. Provisional Patent Application No. 62/132,970 entitled “Apparatus and Methods for Neurological Intervention” and filed Mar. 13, 2015, the contents of which is incorporated herein by reference in its entirety). The probe-side end of the adjustable depth stop 114, in some examples, may be designed to fit over and/or mate with a minibolt mounted to the skull of a patient or a probe driver (as described in U.S. patent application Ser. No. 14/659,488 filed Mar. 16, 2015, U.S. patent application Ser. No. 14/661,194 filed Mar. 18, 2015, and U.S. patent application Ser. No. 14/661,212 filed Mar. 18, 2015, each being entitled “Image-Guided Therapy of a Tissue,” the contents of each of which are incorporated herein by reference in their entireties). For example, the adjustable depth stop 114, as illustrated in the enlarged view of FIG. 1D, can include a latch 119 for releasably attaching the adjustable depth stop 114 to the minibolt or probe driver. In another example illustrated in FIG. 16K an adjustable depth stop may include a thumb screw 1660 for releasably attaching the adjustable depth stop to the minibolt or probe driver. In some embodiments, the adjustable depth stop 114 has a compact width, to provide more clearance within a magnetic resonance (MR) bore to allow easy operation.

Turning to FIGS. 1E and 1F, enlarged views of the adjustable depth stop 114 illustrate both an unlocked position view 122a of FIG. 1E and a locked position view 122b of FIG. 1F. As illustrated in FIG. 1E, in the unlocked position, a probe-end portion 114b of the adjustable depth stop 114 is rotated about 90 degrees offset from a sheath-end portion 114a. When in the unlocked position 122a of FIG. 1E, for example, the adjustable depth stop 114 may be slid along the shaft of the probe 112. If, instead, the adjustable depth stop 114 is mated with another piece of equipment during in situ positioning of the probe 112, the shaft of the probe 112 may be slid within the adjustable depth stop 114 for in-situ repositioning. In another example, while in the unlocked position 122a of FIG. 1E, the adjustable depth stop 114 may be removed from the probe 112. In this manner, the adjustable depth stop 114 may be used with various probes.

FIGS. 16A through 16H illustrate additional depth locking element configurations for use with the variable length probe of FIGS. 1C and 1D. Turning to FIGS. 16A and 16B, a body section 1602a, 1602b of a twist compression jaw style depth locking element 1600a, 1600b includes a vertical opening 1604a, 1604b aligned above a tapered hole or depression 1605a, 1605b as well as a horizontal slot 1606a, 1606b. A nut section 1620a, 1620b fits within the slot 1606a, 1606b, and a jaw section 1608a, 1608b slides through the vertical opening 1604a, 1604b to enter a center opening of the nut section 1620a, 1620b. In use, a probe, inserted through a lengthwise opening 1612a, 1612b of the jaw section 1608a, 1608b while the jaw section 1608, 1608b is nested within the center opening of the nut section 1620a, 1620b via the vertical opening 1604a, 1604b. The jaw section 1608a, 1608b and the nut section 1610a, 1610b include mated features (e.g., threads as illustrated in relation to nut section 1610a of FIG. 16A, slots and grooves as illustrated in relation to nut section 1610b of FIG. 16B) such that, when the nut 1620a, 1620b is turned, the jaw 1608a, 1608b is drawn downwards into the tapered hole or depression 1605a, 1605b of the body section 1602a, 1602b of the jaw style depth locking element 1600a, 1600b. This causes gap portions 1614a-1614c of the jaw 1608a, 1608b to close, narrowing the lengthwise opening 1612a, 1612b of the jaw portion 1608a, 1608b closest to the tapered hole or depression 1605a, 1605b and clamping onto the probe, applying locking friction against the probe.

Turning to FIG. 16C, a hinged compression jaw style depth locking element 1615 includes a body section 1616 and a latch section 1618 which connects at a hinge portion 1618c to a hinge mount 1616c of the body section 1616. In use, a probe is positioned through a probe opening 1618d of the latch section 1618, and the latch section 1618 is locked to the body section 1616 by pressing a protrusion 1618b of the latch section 1618 into a mating depression 1616b of the body section 1616. In doing so, a jaw 1618a of the latch section 1618 is pressed into a depression 1616a of the body section 1616, causing the gaps of the jaw 1618a to close, thus clamping onto and applying locking friction against the probe.

FIG. 16D illustrates a series of positions of a nonconcentric knob style depth locking element 1620 having a body section 1622 with a jaw 1626. The jaw 1626a has an elliptical shape matching an elliptical opening of a knob section 1624a such that, while in an open position 1602a, the knob 1624a fits over the jaw 1626a. In use, a probe can be positioned within the jaw 1626a along a length of the body section 1622a while the knob section 1624a is positioned over the jaw 1626a in the open position 1620a. An operator may identify the open position 1602a due to a bright band (e.g., red) 1628 being visible. Upon positioning of the probe at the desired depth, the operator may twist the knob 1624a, thus aligning the narrow diameter of the elliptical shape of the knob 1624a with the wide diameter of the elliptical shape of the jaw 1626a. This causes the gaps in the jaw 1626a to close, thus clamping onto and applying friction against the probe.

FIGS. 16E through 16G illustrate twin snap style depth locking elements 1630. In a general sense, a twin snap style depth locking element 1630a, 1630b, 1630c functions by positioning a probe through an opening 1642a, 1642b, 1642c and along a length of a body section 1632a, 1632b, 1632c. To releasably attach the twin snap style depth locking element 1630a, 1630b, 1630c to the probe, a locking section 1634a, 1634b, 1634c frictionally snaps onto a mated portion of the body section 1632a, 1632b, 1632c at snaps 1638a. For example, the body section 1644a of the twin snap style depth locking element 1630a of FIG. 16E includes slots 1644a, 1644b for receiving the snaps 1636a of the locking section 1634a.

Twin snap style depth locking elements 1630a and 1630c of FIGS. 16E and 16G additionally include secondary snaps 1640a, 1640c such that the locking section 1634a, 1634c remains connected to the body section 1632a, 1632c when the snaps 1636a, 1636c are disengaged.

To release the probe, for twin snap style depth locking elements 1630a and 1630b of FIGS. 16E and 16F, the operator may squeeze the tabs 1638a, 1638b. For twin snap style depth locking element 1630c of FIG. 16G, the operator may squeeze the locking section at gripping portions 1646 to release.

Turning to FIG. 16H, a Touhy Borst style depth locking element 1650 includes body sections 1652a, 1652b configured to releasably thread together. A probe may be positioned through an opening 1654 within the body section 1652a and along a length of the body sections 1652a, 1652b. Upon threading the body sections 1652a, 1652b together, a deformable (e.g., rubber or silicone) ring 1656 applies frictional pressure to the probe, locking the Touhy Borst style depth locking element 1650 in place. A washer 1658 reduces friction between the deformable ring 1656 and the upper body section 1652a.

Returning to FIGS. 1E and 1F, the adjustable depth stop 114, in some embodiments, includes features for ease of use and precision positioning. In a first example, each of the portions 114a and 114b include one or more grip indents 124 to enable easier grasp of the portions 114a and 114b when twisting the portions 114a and 114b relative to each other to lock and unlock the adjustable depth stop 114. In a second example, sheath-end portion 114a of the adjustable depth stop 114 may include an alignment marker 126 for aligning the adjustable depth stop 114 with the gradations along the shaft of the probe 112 for precision positioning. The alignment marker 126, in the circumstance of a side-firing probe, may be aligned in the direction of firing of the probe. For example, the gradations on the shaft of the probe 112 may be printed along a line positioned in the direction of firing, such that aligning the alignment marker 126 with the line of the gradations aligns a projection 126 of the probe-end portion 114b of the adjustable depth stop 114 with the side-firing direction of the side-firing probe. In an alternative embodiment, as illustrated in FIG. 1D, a directional marker 130 (e.g., line along the sheath-end portion 114a of the adjustable depth stop 114) may identify a side-firing direction of the side-firing probe.

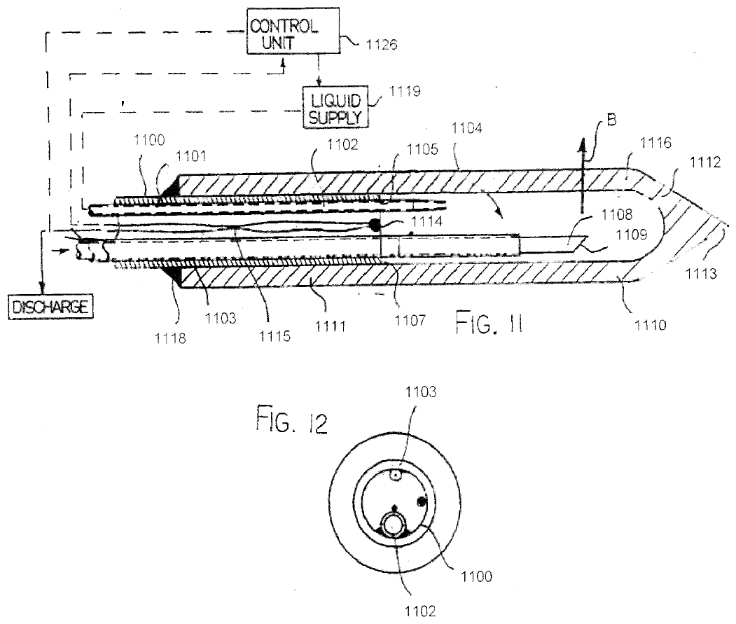

FIG. 1G illustrates an example enlarged cut-away view of the flexible umbilical sheath 116 of the probe apparatus 100 of FIGS. 1C and 1D. The sheath 116, as illustrated, includes a series of windings 132 and an inner conduit 134. The conduit may be used to carrying inputs and outputs (e.g., energy, control signals, thermocouple wires, a deflection wire, cooling gas or fluid, and/or heating gas or fluid) between the probe 112 of FIGS. 1C and 1D and a control unit (not illustrated). In some examples, the conduit 134 may include one or more tubes, lumens, or other divisions to separate various inputs and outputs directed through the conduit 134. For example, as described in greater detail in relation to FIGS. 10 and 12, the conduit may include a number of lumens or tubes to enable cooling fluid supply to and cooling fluid return from a fluid-cooled probe. The windings 132 provide structure to protect the various components within the conduit 134. In some examples, the windings 132 may be configured to supply a particular kink resistance value, torque strength, and/or tensile strength to the flexible umbilical sheath 116. In one embodiment, the coil structure resulted in optimal kink resistance and a tighter coil (as compared with a looser coil) resulted in better column strength. In another embodiment, one or more of the multiple layers has a braided structure. The flexible umbilical sheath 116 may be designed using non-ferro-magnetic materials for MRI compatibility. For example, the windings 132 may be composed of a polymer or poly-vinyl material. In other examples, the flexible umbilical sheath may be composed of PTFE, PEEK, Polyimide, Polyamide and/or PEBAX, and the winding material may include stainless steel, Nitinol, Nylon, and/or PEEK. The materials of the windings 132 and/or the external covering of the flexible umbilical sheath 116 may be selected in part for thermal stability. In the example of a cryotherapy probe such as the probe described in relation to FIG. 4, the materials of the windings 132 and/or the external covering of the flexible umbilical sheath 116 may be selected to withstand extremely cold temperatures without incurring damage during flexing.

FIG. 1H is an enlarged view of the transitional part 120 positioned between the probe 112 and the flexible umbilical sheath 116 of the variable length probe apparatus 110 of FIGS. 1C and 1D. The transitional part 120 may include one or more indents 136 for grasping the transitional part 120 upon positioning or otherwise manipulating the probe 112. Further, the transitional part 120 may include one or more vents 138 for venting return gasses in a gas-heated or JT fluid-cooled probe.

In some embodiments, a shaft portion of the probe 112 including windings 132 (e.g., at least beginning at a point above the gradations and abutting or extending beyond the transitional part 120, if not continuing throughout the entirety of the shaft portion of the probe 112) may be composed of one or more materials selected at least in part for flexibility of the shaft portion such that the shaft portion may bend away from the skull (for that portion of shaft external to the skull of the patient). Probe shaft materials may include, in one example, polyimide for rigidity in a first shaft portion designed for interstitial deployment, and PTFE for flexibility in a second shaft portion interfacing with the windings 132. Additionally, the first shaft portion and/or the second shaft portion may be composed of multiple layers of material, such as polyimide under an etched layer of PTFE, to provide for better bonding characteristics at the interface between the first shaft portion and the second shaft portion.

FIG. 2A illustrates a probe driver 200, which generally includes a commander 202, umbilicals 204, a follower 206, and a position feedback plug 208 that receives position feedback signals, such as potentiometer signals, from the follower 206 via a feedback cable 212. A probe can be inserted into the follower 206, and the follower 206 can control a rotational and longitudinal alignment of the probe.

The probe driver 200 can be mounted to an interface platform, such as the interface platform disclosed in U.S. Pat. No. 8,979,871 to Tyc, entitled “Image-Guided Therapy of a Tissue” and filed Mar. 15, 2013, incorporated by reference in its entirety. The position feedback plug 208, for example, can connect to the interface platform in order to communicate the probe's position to the system. The probe driver 200 is used to rotate or translate (extended or retract) the probe. The probe driver 200 in this illustrated implementation can provide, at a minimum, a translation of 20-80 mm, 30-70 mm, 40-60 mm or 40 mm, with a maximum translation of 60 mm, 80 mm, 100 mm, 120 mm or 60-150 mm. The probe driver 200 in this illustrated implementation can also provide, at a minimum, a rotation of 300°-340°, with a maximum rotation of 350°, 359°, 360°, 540°, 720° or angles therebetween. Included with the probe driver 200 can be a rotary test tool that can be used during a self-test procedure to simulate an attachment of a probe to the follower 206.

The umbilicals 204 may include sheathed wires that independently control rotational and longitudinal motion of a probe or other device held by the follower 206. Independent control of the rotational and longitudinal motion may be provided, for example, by rotating a respective one of the knobs or dials 210 provided at either side of the commander 202. An example structure for the corresponding mechanisms that provide the rotational and longitudinal motion is described and shown in U.S. Pat. No. 8,728,092, entitled “Stereotactic Drive System” and filed Aug. 13, 2009, the entirety of which is incorporated herein by reference.

In various implementations, the probe driver 200 provides full remote control to an operator that is located either: (1) in the proximity of the imaging apparatus and an interface platform that the probe driver is connected to, or (2) in a remote room, such as a control room, at a workstation, where the workstation sends positioning signals to the interface platform to actuate corresponding movements by the commander 202. Full remote control of the probe driver 200 is thus provided, which reduces procedure time.

Turning to FIGS. 2B-2D, a series of views illustrate insertion of a variable length probe apparatus 220 into a probe follower 222. Similar to the variable length probe apparatus 110 described above in relation to FIGS. 1C through 1D, as shown in FIG. 2B, the variable length probe apparatus 220 includes a probe portion 224a integrated with or connected to a flexible umbilical sheath 224b. As illustrated, the probe portion 224a is inserted within an adjustable depth stop 226. The adjustable depth stop 226, for example, may be releasable connected to the probe apparatus 220 or slideably integrated with the probe apparatus 220 (e.g., such that it is not configured for removal from the probe 224).

In an implementation illustrated in FIG. 2C, the adjustable depth stop 226 is separated from the probe portion 224a and aligned with a mating protrusion 228 of the probe follower 222. As illustrated in a mating protrusion 214 of the follower 206 of FIG. 2A, for example, the mating protrusion 228 may be a hollow cylinder configured to mate with a larger diameter cylindrical opening of the adjustable depth stop 226. The probe portion 224a, in this example, would be inserted through the cylindrical opening of the mating protrusion 228 of the probe follower 222. For example, as illustrated in FIG. 2D, the adjustable depth stop 226 is mounted upon the probe follower 222 with the flexible umbilical sheath 224 extending above the adjustable depth stop 226 and the probe portion 224a extended out below the probe follower 222. The adjustable depth stop 226, in this configuration, may be mated with the mating protrusion 228 before or after insertion of the probe portion 224a into the adjustable depth stop 226. As with the follower 206, the probe follower 222 includes both a position feedback cable 230 for communicating position signals as well as umbilicals 232 for receiving positioning commands from a commander unit (not illustrated) such as the commander 202 of FIG. 2A.

Multiple different probes can be utilized and swapped into the follower 206 or follower 222 during treatment so as to provide different therapeutic patterns from different probes. For example, a symmetrical ablation probe can be used, followed by a side-fire (asymmetrical) ablation probe. A diffused tip probe can also be utilized.

A process of advancing probe, asymmetrically treating, measuring, advancing probe and repeating is provided, such that the process does not require the interruption of a user-intervention in the surgical room to change probes or probe position.

Further, in some implementations, the follower 222 includes a low profile design with a short stem to enable wider skull access and/or multiple concurrent probe trajectories. FIGS. 2E through 2G illustrate views 240 of the follower 222 with a low profile design. As shown in FIGS. 2E and 2F, a guide rail portion 242 of the follower 222 may have a short stem 244 for ease of interoperability with various skull-mounted probe introduction equipment, such as a bolt-style probe introduction device 256. The short stem 244, in some examples, may be less than 19 mm or between 19 and 32 mm. In some examples, the length of the short stem 244 is less than or about a third the length of the guide rail 242.

As illustrated in FIG. 2E, the follower 222 is mated with a bolt-style probe introduction device 256. Details regarding a bolt-style probe introduction device are provided in U.S. Provisional Patent Application No. 62/132,970 entitled “Apparatus and Methods for Neurological Intervention” and filed Mar. 13, 2015, the contents of which are hereby incorporated by reference in its entirety. The bolt-style probe introduction device 256 may be locked to the follower 222 via a locking mechanism. As shown, the bolt-style probe introduction device 256 is locked to the follower 222 via a hole in the short stem 244 aligned with a thumb screw 254 inserted through a locking sleeve 248. The locking sleeve 248, as illustrated, has a similar length as the short stem 244. In other implementations, the locking sleeve 248 may include an elongated design, for example based upon mating requirements with particular skull-mounted probe introduction equipment. Although described in relation to the thumb screw 254, alternatively, in some examples, a pin and groove design, locking teeth, or clamp lock may be used to lock the bolt-style probe introduction device 256 to the follower 222.

The locking sleeve 248 in some implementations, includes a poka-yoke design 250 such that the locking sleeve 248 naturally aligns with a thread opening 252 of the locking sleeve 248 with the hole 246 of the short stem 244.

The follower 222 with low profile design may be manufactured of thermal imaging compatible materials, such as MRI-compatible materials. Additionally, the locking sleeve 248 and/or thumb screw 254 may be manufactured of thermal imaging compatible materials. In some embodiments, at least a portion of the follower 222 may include imaging system-identifiable material such as a thermal imaging identifiable fiducial marker for use in identifying the location and/or orientation of the follower 222 through medical imaging or other means such as RF proximity systems. In a particular example, a fiducial marker 258, as illustrated in FIG. 2G, may be positioned upon a bottom portion of the guide rail 242. In addition to or in lieu of a fiducial marker 258, in other embodiments, the follower 222 with low profile design may include electronic (e.g., RFID-radio frequency identification) markers and/or visual markers compatible with visual imaging systems.

FIG. 3A illustrates an example temperature modulation probe 300 for modulated application of thermal therapy and cryotherapy using both a thermal therapy-generating element and a cryotherapy-generating element disposed within the temperature modulation probe 300. Further, the temperature modulation probe 300 may provide a single tool capable of spanning a wide range of potential thermal output from cryotherapy-induced cellular death to thermal therapy-induced cellular death. In a particular example, the temperature modulation probe 300 may be capable of reaching a substantial range, if not all, of the temperatures and effects described in relation to FIGS. 7A and 7B. In a modulation therapy use, the temperature modulation probe 300 supplies a modulated temperature output pattern to a target tissue, varying between a warmer temperature applied at least in part by the thermal therapy-generating element and colder temperature applied at least in part by the cryotherapy-generating element. Treatments enabled by the temperature modulation probe 300 may include treatments that create temporary or permanent physical-biological effects to tissue including freezing freeze-thawing, hyperthermia, coagulation, and/or vaporization of tissues. The temporary or permanent physical-biological effects can include alterations in biological function of cells, tissues, and/or body fluids. In a particular example, the treatment may cause the cells, tissues, and/or body fluids to be more receptive or sensitive to additional therapies or manipulations such as, in some examples, drug therapy, radiation therapy or chemotherapy. In a further example, the treatment may cause hemostasis, reduction or dissolution of thrombi or emboli, alteration of functional membranes including the blood-brain barrier, and/or renal filtration. The physical-biological effects may be caused directly by temperature change to the cells, tissues, and/or body fluids or indirectly (e.g., downstream) from the temperature change, such as alterations in heat shock proteins or immune reaction or status. The temperature modulation probe 300 may be designed for insertion into a body cavity, insertion into vascular system, or interstitial deployment.

As illustrated, the temperature modulation probe 300 includes a laser 302 with a side-firing tip 304 as a thermal-therapy generating element. The side-firing tip 304 may further include a side-firing diffuser capable of focal ablation with a lower power density. Examples of side-firing tips and various diffusing patterns for side-firing probes are described in U.S. Pat. No. 8,979,871 to Tyc, entitled “Image-Guided Therapy of a Tissue” and filed Mar. 15, 2013, incorporated by reference in its entirety.

Although the temperature modulation probe 300 is illustrated with the side-firing laser 302, in other embodiments, the temperature modulation probe 300 includes a forward firing probe tip, such as a probe tip 322 illustrated in relation to the laser probe 320 of FIG. 3B.

In further embodiments, rather than or in addition to the laser fiber 304, the temperature modulation probe 300 may include one or more ultrasound elements and/or ultrasonic transducers capable of focal or diffuse heating using HIFU. The ultrasonic beam of a HIFU probe can be geometrically focused (e.g., using a curved or flat ultrasonic transducer element or lens) or electronically focused (e.g., through adjustment of relative phases of the individual elements within an array of ultrasonic transducers). In an ultrasonic transducer array, the focused beam can be directed at particular locations, allowing treatment of multiple locations of a region of interest without mechanical manipulation of the probe. The depth of treatment can be controlled by adjusting the power and/or frequency of the one or more transducers of the HIFU probe. Example HIFU probes are described in U.S. patent application Ser. No. 14/661,170, entitled “Image-Guided Therapy of a Tissue” and filed Mar. 18, 2015, the contents of which are incorporated by reference herein in its entirety.

In additional embodiments, the temperature modulation probe 300 may include a microwave, RF, heating gas, heating fluid, or electrical heat thermal therapy-generating element in lieu of or in addition to the laser 302. A heating fluid thermal therapy-generating element, for example, may circulate a fluid such as helium or hydrogen. Each of the at least one thermal therapy-generating element may be configured to emit thermal energy in a side-firing, focal, or diffuse manner. In a particular example, a temperature modulation probe includes a circumferentially emitting thermal therapy-generating element. The temperature modulation probes of the present disclosure may be designed for insertion into a body cavity, insertion into vascular system, or interstitial deployment.

The temperature modulation probe 300 further includes a cryotherapy-generating element 306 (e.g., cooling gas, cooling fluid, etc.) for supplying cryotherapy to the effected tissue. In some examples, the cryotherapy-generating element 306 may include a flow of fluid such as gaseous carbon dioxide, liquid nitrogen, or liquid or gaseous forms of argon. The supplied fluid/gas may utilize Joule-Thomson cooling via Joule-Thomson expansion. As described in relation to FIG. 4, for example, a cooling fluid delivery tube with reduced diameter aperture or orifice may be used to deliver a fluid or gas at a predetermined pressure. The restricted orifice or aperture of the fluid delivery tube may be a venturi outlet having a cross-sectional area smaller than a main-body of the cooling tube. Gas or fluid exiting the reduced diameter aperture or orifice, via Joule-Thomson expansion, will expand into an expansion chamber to provide a cooling effect to the tip of the probe 300. Fluids may be provided as a liquid via the fluid delivery tube and, upon expansion into the expansion chamber, the fluid may form a gas, going through an adiabatic gas expansion process through the restricted orifice into the expansion chamber to provide the cooling effect. Alternatively, cooling fluids which do not expand but rather circulate can also be used.

The cryotherapy-generating element 306 may be configured to emit cryotherapeutic energy in a focal, circumferential, or diffuse manner. In some configurations, a probe may include multiple cryotherapy-generating elements 306 and/or multiple supply orifices for a single cryotherapy-generating element 306 to provide a particular deployment shape or pattern. In some examples, multiple orifices may be arranged in a line, a cluster, and/or a circular pattern to form a longer and/or wider cooling pattern at a distal tip of the temperature modulation probe 300. For example, arranging several orifices along the probe tip may provide an ellipsoidal three-dimensional cooling zone extending from the distal tip of the temperature modulation probe 300. In some embodiments, the multiple orifices may be provided via multiple perforations or other openings of a distal tip of the cryotherapy-generating element 306. In other embodiments, each orifice may include a separate venturi nozzles or other release valve, such that the cryotherapy-generating element 306 may produce two or more delivery patterns (e.g., based upon which release valve(s) of a number of release valves is placed in an open position). A controller, such as software and/or firmware, may control the patterning of a multi-nozzle, multi-pattern cryotherapy-generating element 306.

The cryotherapy-generating element 306, in some embodiments, is designed for compatibility with the thermal therapy-generating element. For example, the fluid or gas supplied by the cryotherapy-generating element 306 may be selected to avoid interference with the transmission of heat energy by the thermal therapy-generating element (e.g., will not alter laser light attenuation due to absorption by the coolant fluid).

In some implementations, an energy output pattern of the temperature modulation probe 300 includes simultaneous activation of at least one cryotherapy-generating element and at least one thermal therapy-generating element. For example, emissions of both a cryotherapy-generating element and a thermal therapy-generating element may be combined to refine control of temperature emission of the temperature modulation probe. Modulation may further be achieved by varying output of at least one cryotherapy-generating element relative to at least one thermal therapy-generating element. For example, laser power, pulse timing, RF cycling, HIFU element frequency and/or power, fluid or gas pressure, fluid or gas temperature, and/or flow rate may each be varied to obtain temperature modulating output and/or controlled temperature output.

Temperature production of the temperature modulation probe 300, in some implementations, is refined based upon temperature measurements obtained by a temperature sensor element 308 such as the temperature sensor element 408 described in relation to the probe 400 of FIG. 4.

In some implementations, an on-board processor of the temperature modulation probe 300 controls temperature modulation. Temperature modulation control can be managed by the temperature modulation probe 300, in one example, based upon activation of one or more pre-set patterns programmed into the temperature modulation probe 300. In another example, the temperature modulation probe may maintain or vary a probe temperature (e.g., at the tip of the temperature modulation probe 300) by monitoring temperature measurements gathered by the temperature sensor element 308.

A controller, such as a workstation or computing device, in some implementations, manages temperature modulation of the temperature modulation probe 300 through successively (or concurrently) activating cryotherapy-generating element(s) 306 and/or thermal therapy-generating element(s) 302 via remotely supplied commands. In additional implementations, the controller may manage temperature modulation of the temperature modulation probe 300 through modifying fluid or gas flow rates, fluid or gas temperatures, laser power and pulse timing, etc.

Further, in some implementations, the controller may manage temperature modulation by modifying output of a fluid or gas within the temperature modulation probe through remotely controlling a release aperture through which the fluid or gas is delivered. For example, as discussed in relation to the probe 400 of FIG. 4, an aperture at the orifice of the gas or liquid injection tube may be remotely controlled.

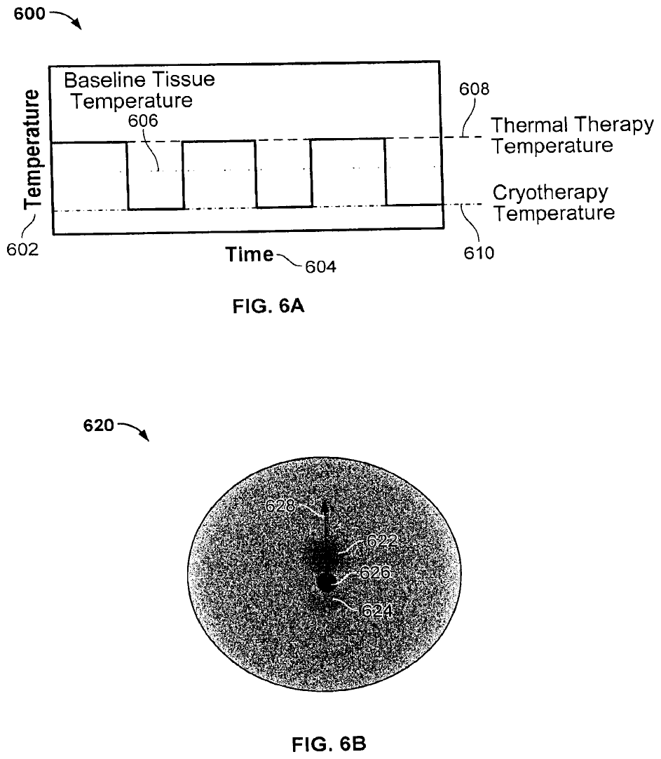

Turning to FIG. 6A, a graph 600 illustrates an example modulation pattern for temperature modulation therapy of a tissue using a temperature modulation probe, such as the probe 300 of FIG. 3A. As illustrated, temperature 602 is modulated over a period of time 604 between a thermal therapy temperature 608 above a baseline tissue temperature 606 and a cryotherapy temperature 610 below the baseline tissue temperature 606. Although the time periods of the thermal therapy temperature, in the illustration, appear to be about one third greater than the time periods of cryotherapy temperature, the graph is for illustrative purposes only. In other embodiments, the relative durations of thermal therapy temperature and cryotherapy temperature may vary. In further embodiments, a thermal pattern may include three or more temperature levels or programmable single or multiphase temperature slope profiles. Although illustrated as being modulated about the baseline tissue temperature 606, in yet other embodiments, a thermal pattern may include two or more temperature levels, all above the baseline tissue temperature 606 or, conversely, two or more temperature levels, all below the baseline tissue temperature 606.

The thermal therapy temperature 608 of the example modulation pattern may be applied to the tissue by one or more thermal therapy-generating elements of a temperature modulation probe. In alternative embodiments, the thermal therapy temperature 608 is applied to the tissue by one or more thermal therapy-generating elements of the temperature modulation probe during simultaneous application to the tissue by one or more cryotherapy-generating elements of the temperature modulation probe. In one example, simultaneous application may reduce an overall temperature of the therapy, for example maintaining thermal output in a range of reversible cellular damage or other non-ablative therapy. In another example, simultaneous application may be used to protect tissue within close proximity to the probe from undesirable damage (e.g., charring) while using the thermal therapy-generating elements to cause irreversible cellular damage (e.g., during laser ablation application). In a further example, simultaneous application may provide less aggressive temperature ranges for therapy applied in pediatric cases or where boundaries of sensitive cells or tissues are in close proximity to the intended target.

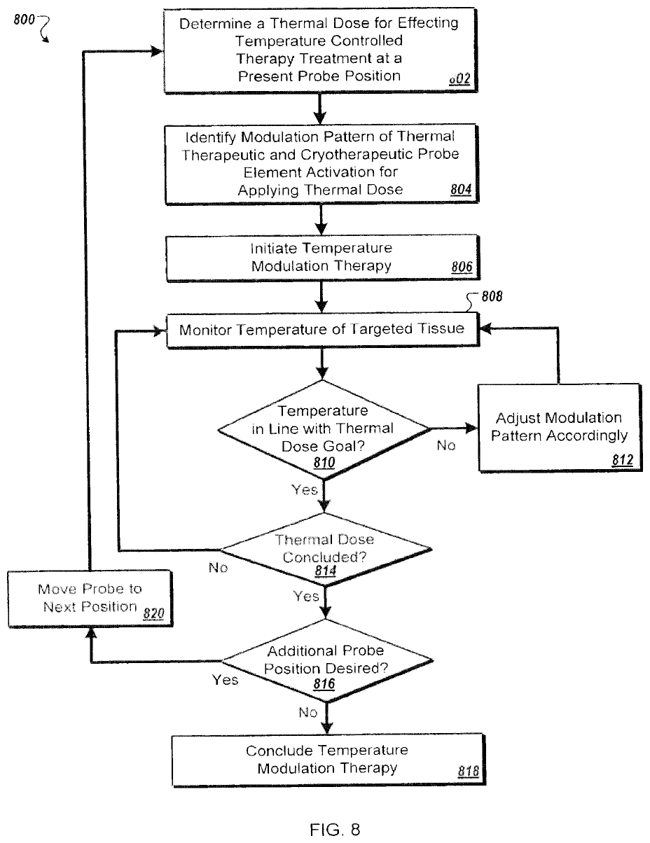

The cryotherapy temperature 610 of the modulation pattern may be applied to the tissue by one or more cryotherapy-generating elements of the temperature modulation probe. Application of a temperature modulation enabled therapy using a temperature modulation probe such as the probe 300 is described in greater detail below in relation to FIG. 8. In alternative embodiments, the cryotherapy temperature 610 is applied to the tissue by one or more cryotherapy-generating elements of the temperature modulation probe during simultaneous application to the tissue by one or more thermal therapy-generating elements of the temperature modulation probe. In one example, simultaneous application may increase an overall temperature of the therapy, for example maintaining thermal output in a range of reversible cellular damage (e.g., when causing hypothermic stress). In another example, simultaneous application may be used to protect tissue within close proximity to the temperature modulation probe from undesirable damage while using the cryotherapy-generating elements to cause irreversible cellular damage (e.g., avoiding intracellular ice development).

FIG. 6B illustrates example effects upon a tissue produced by a temperature modulation probe. Turning to FIG. 6B, a diagram 620 illustrates a heat-exposed region of the tissue 622 (illustrated in red) and a cold-exposed region of the tissue 624 (illustrated in blue) caused by a temperature modulation probe disposed at point 626. The temperature modulation probe 626, for example, includes a side-firing thermal-therapy generating element firing in the direction of arrow 628. The cryotherapy-generating element of the temperature modulation probe may be a diffuse element, causing cryotherapeutic temperatures surrounding the axis of the temperature modulation probe. In a particular example, the effects illustrated in FIG. 6B may be caused by constant diffuse application using a cryotherapy-generating element while modulating application of the thermal therapy-generating element.

FIG. 8 is a flow chart of an example method 800 for effecting a temperature modulation therapy using a temperature modulation probe including at least one thermal therapy-generating element and at least one cryotherapy-generating element. The method 800, for example, may be performed using the temperature modulation probe 300 described in relation to FIG. 3A. Aspects of the method 800 may be performed with automated equipment for controlling operation of a temperature modulation probe, such as the commander and follower described in relation to FIG. 2. Aspects of the method 800, for example, may be performed on a workstation or other computing device in communication with the automated equipment for controlling operation of the temperature modulation probe. The workstation or other computing device, for example, may be configured to transmit position control signals to the automated equipment for controlling operation of the temperature modulation probe and/or energy control signals to one or more energy sources, coolant sources, or heat-producing sources delivering energy, fluid, or gas to the temperature modulation probe. The workstation or other computing device, in one example, may be configured to process a sequence of energy, fluid, and/or gas control signals to effect a temperature modulation therapy to the tissue with the temperature modulation probe. Further, the workstation or other computing device may be configured to analyze temperature data received from the temperature modulation probe, a temperature sensor deployed within or near the tissue, and/or a thermal imaging module. Based upon the temperature data analysis, the workstation or other computing device may be configured to modify, suspend, or complete the temperature modulation therapy.

In some implementations, the method 800 begins with determining a thermal dose for effecting temperature controlled therapy treatment at a present position of the temperature modulation probe (802). The present position, for example, corresponds to a particular region of interest of tissue. The thermal dose may include a goal temperature, a goal thermal dose profile, or a goal energy dose profile. Thermal dose profiles, with respect to a specified time period, can include one or more temperatures or temperature gradients for effecting treatment to the particular region of interest. The thermal dose profiles and/or the temperature gradients may permit the determination of an extent of cellular damage in the targeted tissue area and/or other effects upon the targeted tissue area occurring as a result of the temperature modulation therapy. Energy dose profiles may describe energy emissions, over time, which are calculated to cause a particular thermal effect upon the targeted tissue. The thermal dose, for example, may correspond to a desired effect upon the targeted tissue, such as altering normal biological function (e.g., electrical impulse carrying capacity, cytoplasmic enzyme activity, etc.), altering abnormal biological function (e.g., oncogenes, etc.), or influencing cellular activity by an external agent (e.g., drug, chemical, biochemical, etc.). A workstation or computing device, in some embodiments, may determine the thermal dose based upon properties of the region of interest, the desired effect upon the targeted tissue, and/or a type of secondary treatment to be applied at the region of interest (e.g., drug, chemical, biochemical, radiation, etc.).

In some implementations, a modulation pattern of thermal therapy-generating element activation and cryotherapy-generating element activation is identified for applying the thermal dose (804). The modulation pattern may be pre-programmed (e.g., corresponding to the desired thermal dose and/or desired effect) or independently calculated (e.g., using particular properties of the region of interest, desired effect upon the targeted tissue, and/or type of secondary treatment to be applied at the region of interest). Further, the modulation pattern may be static (e.g., applied during the entire temperature modulation therapy) or dynamic (e.g., capable of real-time adjustment based upon temperature monitoring of the tissue during temperature modulation therapy). In other implementations, the modulation may be user-controlled (e.g., user entered and/or user-activated switching between thermal therapy-generating element(s) and cryotherapy-generating element(s).

In some implementations, temperature modulation therapy is initiated (806). In some examples, an operator may physically activate (e.g., depress a button, switch a control) or electronically activate (e.g., via a graphical user interface control element) functionality of the temperature modulation probe. In a particular example, an operator at a workstation may activate temperature modulation therapy by depressing a foot pedal operatively connected to the workstation to activate the first energy or temperature emission of the modulation pattern (e.g., via a corresponding thermal therapy-generating element or cryotherapy-generating element of the temperature modulation probe).

In some implementations, temperature(s) of the targeted tissue is monitored (808). Initiating temperature modulation therapy may further include initiating thermal monitoring of the targeted tissue. In other embodiments, thermal monitoring is initiated separately from temperature modulation therapy. For example, thermal monitoring may begin prior to temperature modulation therapy to establish a baseline temperature and verify functionality of temperature monitoring equipment prior to activation of temperature modulation therapy. One or more temperatures within the target tissue may be monitored. For example, if the thermal dose included a temperature profile corresponding to a desired temperature gradient, multiple temperatures at multiple locations within and/or abutting the target tissue may be monitored. In one example, temperature monitoring includes receiving temperature data from a temperature sensor element built into the temperature modulation probe. In another example, temperature monitoring includes receiving temperature-sensitive imaging data from an imaging device capturing images including the targeted tissue. In a particular example, utilizing MRI imaging in real time guidance may provide controlled accuracy, while contemporaneous thermography may provide accurate temperature information in determining whether a tissue has achieved a goal temperature or temperature profile to producing a desired therapeutic effect.

In some embodiments, temperatures are monitored throughout the temperature modulation therapy. In other embodiments, temperatures are monitored periodically during temperature modulation therapy. For example, due to potential interference with a particular style of emission element, temperature monitoring may be activated during de-activation of that particular style of emission element. In a particular example, temperature monitoring may be activated during emission of the cryotherapy-generating element of the temperature modulation probe but suspended during emission of an RF style thermal therapy-generating element of the temperature modulation probe. Temperature monitoring of a tissue is discussed in greater detail in U.S. Pat. No. 8,979,871 to Tyc, entitled “Image-Guided Therapy of a Tissue” and filed Mar. 15, 2013, incorporated by reference herein in its entirety.

In some implementations, if the temperature(s) is not in line with the thermal dose goal (810), the modulation pattern is adjusted accordingly (812). Depending on the ability of the tissue or surrounding environment to absorb, conduct, or moderate heating or cooling, for example, the actual temperature changes within the target tissue may fail to correspond with the planned tissue temperature(s). For example, temperature(s) in the target tissue may be lower or higher than desired according to the thermal dose. In this circumstance, the modulation pattern driving emission of the temperature modulation probe may be adjusted to effect the desired change (e.g., increase or decrease in target tissue temperature(s)). The workstation or other computing device, for example, may adjust modulation parameters based upon analysis of temperature data.

Temperature modulation therapy may be suspended, in some embodiments, prior to modulation pattern adjustment. For example, if tissue temperatures are outside of a range associated with a desired therapeutic effect (e.g., moving from temperatures associated with reversible cellular damage to temperatures associated with cellular death), temperature modulation therapy may be temporarily suspended while adjusting the temperature modulation pattern. In other embodiments, temperature modulation therapy may continue during the adjustment period.

In some implementations, if the temperature is in line with the thermal dose goal (810), it may be determined that the thermal dose is concluded (814). For example, the thermal dose may correspond to a particular temperature (or temperatures) for a particular period of time or a series of such temperature profiles or temperature gradients. Temperature monitoring may include analyzing historic temperature measurements of the target tissue to verify that the target tissue has reached a particular temperature (or a particular temperature range) for a particular length of time. In other embodiments, determining conclusion of thermal dose includes analyzing the target tissue for evidence of a desired physical-biological effect. The evidence, in one example, may be derived through analysis of image data. In another example, the evidence may be derived through an invasive analysis element, such as the recording elements described in relation to FIGS. 5A through 5H.

In some implementations, if the thermal dose is concluded (814), and an additional probe position is desired (816), the probe is moved to a next position (820). In some embodiments, a rotational and/or linear position of the probe may be adjusted by translating or rotating the probe via automated probe manipulation equipment, such as the commander and follower described in relation to FIG. 2. For example, a workstation or other computing device may direct the automated probe manipulation equipment to reposition the probe to a new rotational and/or linear position. In other embodiments, the probe position is adjusted manually. In one example, the workstation or other computing device may present one or more recommended adjustments (e.g., upon a graphical user interface) for review by a medical professional, and the medical professional may follow the instructions to manually adjust the position of the probe. If the probe position is automatically adjusted, in some embodiments, temperature modulation therapy may continue during repositioning. For example, the temperature modulation pattern may remain active while the probe is translated to a next position. In other embodiments, energy output of the temperature modulation probe may be terminated prior to repositioning.

After adjusting the probe position, in some embodiments, the method 800 returns to determining a thermal dose for effecting temperature controlled therapy treatment at the present probe position (802) and identifying a modulation pattern (804). In other embodiments, the method 800 may continue to use the previously determined thermal dose and/or modulation pattern while proceeding with temperature modulation therapy (806) at the next probe position. The method 800 may thus continue until an entire volume of interest within the targeted tissue has been treated, at which time temperature modulation therapy is concluded (818).

Although described as a particular series of steps, in other implementations, the method 800 may include more or fewer steps. For example, after determining that the thermal dose is concluded (814), and prior to determining whether an additional probe position is desired (816), an additional therapy may be applied to the temperature modulation therapy-treated tissue at the present position. For example, upon preparing the tissue for increased sensitivity to a particular drug or radiation treatment via temperature modulation therapy, the method 800 may include deploying the particular drug or radiation treatment at the present position prior to moving the probe to the next position (820). Secondary treatment, in this example, may be performed by the same probe or an additional instrument. For example, the temperature modulation probe may be retracted into a shared sheath, while a pharmaceutical therapy instrument is supplemented at the same position for performing the secondary treatment. In this example, moving the probe to the next position 820 may involve moving the sheath containing both the probe and the secondary instrument to the next position.

Additionally, in other implementations, steps of the method 800 may be performed in a different order. For example, as discussed above, temperature monitoring of the targeted tissue (808) may begin prior to initiating temperature modulation therapy (806).

As illustrated in FIG. 3B a focal laser probe 320 includes a short lens region 322 (e.g., clear capsule) for focusing the laser. The lens 322, in some examples, may be composed of ceramic polymer, or quartz. The focal laser probe 320 may be used for providing focal thermal therapy through at least one of ablation, coagulation, cavitation, vaporization, necrosis, carbonization, and reversible thermal cellular damage. The focal laser probe 320, for example, may be used to provide focal ablation with minimal edema for minimally invasive neurosurgical applications. The focal ablation provides precision to protect surrounding tissues during thermal therapy, while the minimal edema encourages immediate therapeutic benefit. The focal laser probe 320, for example, may be included as an alternative embodiment of various probes described herein (e.g., probe 300 of FIG. 3A, probe 900 of FIG. 9, the probe of FIG. 11, probe 1300 of FIG. 13A, probe 1400 of FIG. 14A or probe 1500 of FIG. 15A) by exposing only a forward directed tip of the laser fiber and shortening the capsule portion of the respective probe to avoid stray energy transmission, for example due to internal reflections. Additionally, the shortened capsule portion may be easier to manufacture, reducing costs of the focal laser probe.

In designing the focal laser probe 320, a tip portion 324 of the lens 322, in some implementations, is shaped to best direct the focal energy of the tip of the laser fiber. For example, the tip portion 324 may be substantially flat in shape (e.g., potentially with some rounding to enable better penetration of the probe into the tissue region). In another example, the tip portion 324 may be substantially rounded to encourage a substantially even diffuse pattern of focal energy throughout the entire tip portion 324 of the probe 320. In designing the focal laser probe 320 to direct energy from the tip portion 324 of the lens 322 to create ablation zones directly ahead of the probe rather than from the side, the tip portion of the laser fiber itself can be plain (e.g., flat) cut so the energy directly exits the tip of fiber along its axis.

FIG. 4 illustrates an example probe 400 configured for cryotherapy (cryogenic therapy) including at least cryoablation. The probe 400 may include an injection tube 404 for delivering a refrigerant 402 to a tip region 414 of the probe 400. The probe 400 for example, may employ Joule-Thompson cooling to provide a range of temperatures to the tip region 414. For example, The fluid supply to the injection tube 404, in some embodiments, is controlled by a control unit to generate a predetermined pressure within the fluid supply to the injection tube 404 which can be varied so as to vary the flow rate of the refrigerant 402 into an expansion area of the probe 400, thus varying the temperature at the exterior of the tip region 414 abutting the tissue.

In some implementations, the probe 400 may include multiple cryotherapy-generating elements and/or multiple supply orifices for a single cryotherapy-generating element to provide a particular deployment shape or pattern. In some examples, multiple orifices may be arranged in a line, a cluster, or a circular pattern to form a longer and/or wider cooling pattern at a distal tip of the probe 400. In some embodiments, the multiple orifices may be provided via multiple perforations or other openings at an aperture of the injection tube 404. In other embodiments, each orifice may include a separate nozzle (e.g., venturi nozzle, etc.) or other release valve, such that the probe 400 may produce two or more delivery patterns (e.g., based upon which release valve(s) of a number of release valves is placed in an open position). A controller, such as software and/or firmware, may control the patterning of a multi-nozzle, multi-pattern probe 400.

Further, the sizing and/or shape of the orifice(s) may be selected to produce differing effects, such as expanding or contracting (e.g., focusing) deployment of cryotherapy energy. For example, in the circumstance where multiple perforations or release valves are arranged along a length of a tip portion of the probe, the perforations or release valves may include a vent shaping to direct the cryotherapeutic stream in a direction other than perpendicular to the surface of the injection tube 404 at which the particular orifice is positioned.

In some embodiments, the aperture at the orifice of the injection tube 414 and/or individual release valves arranged upon may be mechanically and/or electrically adjustable to vary flow rate of the refrigerant within the probe 400 itself. Further, the size or patterning of the aperture may be adjusted to vary focal diameter of the refrigerant 402 at the tip region 414 of the probe 400, for example by maintaining a flow rate (e.g., via an external control unit) while adjusting the outlet available to the refrigerant at the tip of the injection tube 414. The aperture, in some examples, may include an adjustable valve or a porous plug. In a particular example, to avoid use of components which may interfere with the imaging system and/or energy producing elements of the cryoablation probe 400, a deflection wire 410 may be used to mechanically manipulate the aperture of the orifice of the injection tube 404. In other embodiments, the deflection wire 410 is used to direct the tip of the injection tube 414 in an offset direction (e.g., in the circumstance of a flexible probe body).

A vacuum return lumen 406, in some embodiments, allows a return cycle of the refrigerant from the expansion region of the probe 400. Thus, the refrigerant is pumped through the injection tube 404 and escapes from the end of the injection tube 404 into the tip region 414 of the probe 400, and then the evaporated refrigerant is returned through the vacuum return lumen 406. From the vacuum return lumen 406, the evaporated refrigerant may be released to the atmosphere or connected to a return tube (not illustrated) to direct the evaporated refrigerant to a return collection. In a particular example, as illustrated in FIG. 1H, the evaporated refrigerant may be released to the atmosphere via the vent 138 of the transition portion 120. In other embodiments, a return tube (not illustrated) is connected to a return collection for the refrigerant. The refrigerant, in some examples, may be liquid nitrogen allowed to expand to nitrogen gas at cryogenic temperatures or liquid argon allowed to expand to argon gas at cryogenic temperatures.

In some implementations, the probe 400 includes a temperature sensor 408 such as one or more thermocouple wires or a fiber optic thermometer. The temperature data generated by the temperature sensor 408, for example, may be provided to a control unit (not illustrated) for monitoring temperature at the tip region 414 of the probe 400. For example, prior to initiation of therapy, the temperature sensor 408 may collect temperature data representing a temperature of tissue proximate to the tip region 414 of the probe 400 and provide the temperature data to the control unit for use as a baseline temperature in monitoring temperature changes during therapy. The control unit, responsive to temperature fluctuations may modulate delivery of the refrigerant 402 to maintain (or controllably fluctuate) a temperature at the exterior of the tip region 414.

In some implementations, the probe 400 includes at least one electroencephalography (EEG), stereo EEG (SEEG), or electrocardiography (ECG) recording element (e.g., wire, electrode, coil, etc.) 412 for monitoring biological rhythms or electrical signals or activity (e.g., within the brain) during positioning of the probe 400 and/or during cryotherapy using the probe 400. The pulse data generated by the recording element 412, for example, may be provided to a control unit. Uses for the data collected by the recording element 412 are described in relation to FIGS. 5A through 5E, below.

FIGS. 5A through 5E illustrate example options for incorporation of one or more recording elements with a thermal therapy, cryotherapy, or temperature modulation therapy probe such as the probe 300 described in relation to FIG. 3A or the probe 400 described in relation to FIG. 4. FIGS. 5F through 5H, further, illustrate example options for designing a recording instrument including at least one recording element. In some examples, a recording element may include an electrocardiography (ECG) recording element, an electroencephalography (EEG) recording element, and/or stereo EEG (SEEG) recording element.

In some embodiments, a recording element is incorporated into a recording instrument or therapeutic probe for recording signals used to detect abnormal neurological, cardiac, spinal, and/or other in vivo tissue response signals. The recording instrument or probe, for example, may include the ability to electrically stimulate nearby tissue then detect abnormal signals issued by the tissue responsive to stimulation. In another example, the recording instrument may include the ability to thermally stimulate nearby tissue then detect abnormal signals issued by the tissue responsive to stimulation. Detection may involve sub chronical recording and stimulation to detect abnormal signals.

A recording element incorporated into a therapeutic probe, in some embodiments, is used for lesion localization and assessment at the time of cryotherapy or thermal therapy. In lesions without sclerosis, the lesion may not be visually detectable. As such, to appropriately position a probe including a recording element, the recording element may be used detect a signal pattern indicative of the position of the lesion.

In some embodiments, a recording element incorporated into a therapeutic probe is used for detection of critical structures surrounding a target tissue area prior and/or during cryotherapy or thermal therapy. For example, identification of blood vessels, nerves, functional motor strip within motor cortex, corticospinal track and other critical neural pathways.

In some embodiments, a recording element is incorporated into a recording instrument or probe with a cryogenic energy element and/or thermal energy element for thermally stimulating the tissue. For example, the tissue proximate to the recording instrument or probe may be cooled using a cryogenic energy element to modify signal activity, such as causing brain signal activities detected by an EEG recording element to go into a hibernation pattern (e.g., at less than 10° C.). Through warming (naturally or aided with a thermal energy element), the “wake-up” patterns triggered within the tissue may be detected by the EEG recording element, thus allowing detection of a signal pattern indicative, in some examples, the position of a lesion or an epileptogenic region (e.g., epilepsy onset spot). In another example, the tissue proximate to the recording instrument or probe may be warmed using a thermal energy element to modify signal activity of the effected tissue. In further examples, tissue temperature may be modulated (e.g., cooled and warmed, or vice-versa, two or more times) while identifying epilepsy onset spots or lesions.

In some embodiments, a set of recording instruments, each including at least one cryotherapy element and/or thermal therapy element, may be deployed in the brain of a patient to map a signal network of activity within the patient's brain. The signal activity, for example, may be mapped to geographic locations within the patient's brain in the signal network to determine one or more regions or zones associated with symptom activity or other evidence of unwellness conditions. The set of recording instruments, for example, may feed to a control system including software for manual and/or automatic control of each of the recording instruments to effect collection of signals to create the signal network. The signal network, for example, may illustrate signal activity during one or more of a resting state and an episode (e.g., seizure) state. The control system may be connected in a wired or wireless manner to each of the set of recording instruments. The control system may be a portable control system, such as a handheld computing device, to allow some patient mobility during monitoring using the set of recording instruments. Monitoring, for example, may include recording of data sets for creation of one or more signal networks over a span of hours or days. The control system may include a user interface for manual adjustment of each of the set of recording instruments, such as activation or deactivation of the cryotherapy/thermal therapy element. The control system may include a wireless computing system remotely located from the set of recording instruments, for example to collect and analyze signals captured by the set of recording instruments. The control system may, in part, include a server-based storage and analysis system accessible via a network such as the Internet.

In some embodiments, a signal network or sets of signal networks collected from a patient may be analyzed by the control system or another data analysis system to identify patterns associated with an unwellness condition, such as patterns indicative of Alzheimer's. In another example, a signal network may be analyzed to identify epileptic seizure activity zones. Further to this example, upon detection of pre-seizure activity or seizure activity within the brain (e.g., using a set of recording instruments and/or an additional monitoring system), the control system (or a user thereof) may cause one or more recording instruments of the set of recording instruments to cool surrounding tissue to suppress signals captured within those regions. In this manner, signals proximate to the cooled region and/or remote from the cooled region may be collected and analyzed without interference of additional signal activity (e.g., due to temporary “hibernation” of the cooled region).

A recording element, in some implementations, is incorporated into a recording instrument or therapeutic probe for detecting permanent or reversible blood brain barrier (BBB) changes (e.g., effected between 40 and 45° C., for example at about 43° C.). For example, the recording element may detect BBB disruption by identifying Gadolinium presence. In a particular example, electro-chemical sensing of a total amount of charge, electrical current, or other property changes (e.g. protein, chemical, drug, marker concentrations) generated by the BBB disruption may be measured to detect the BBB disruption event.

In some embodiments, a recording element of a therapeutic probe provides monitoring during functional neurosurgery. In the example of epileptic symptoms, the recording element may be used to confirm positioning of therapeutic energy for treatment of seizure activity. In another example, a recording element may be used to confirm disruption of the blood-brain barrier. In an additional example, a recording element may be used for monitoring while performing an operation or other therapy, such as monitoring patient biorhythms or electrical activity in the brain and adjusting or suspending therapy if an abnormal event is detected. In another example, the recording element may be used to apply local tissue stimulation responsive to detection of an abnormal event to regulate cellular behaviors during treatment. In particular, the recording element may effect deep brain stimulation during a neurosurgical operation. Further to this example, the recording element may be used to verify efficacy of the local tissue stimulation in modifying the abnormal signals previously detected.

Turning to FIG. 5A, in some implementations, multiple recording elements 502 may be arranged in ring formation along the outer shaft of a probe 500. The recording elements 502, for example, may include individual micro electrodes. In some examples, the recording elements 502 are EEG elements or ECG elements. Although illustrated as including three recording elements 502a-502c, in other embodiments, the probe 500 may include more or fewer recording elements 502, such as five or ten recording elements 502. As illustrated, the recording elements 502 are positioned at a distance from an emission region 504 (e.g., capsule, lens, or other heat, cool, and/or energy emitting portion of the probe 500). In one example, the recording elements 502 may be separated from the emission region 504 to avoid interference with the cryotherapy element(s) and/or thermal therapy element(s) of the probe 500.

Turning to FIGS. 5B and 5C, in some implementations, a probe 510 may be inserted into a guide sheath or sleeve incorporating one or more recording elements. As illustrated in FIG. 5B, the probe 510 is inserted through a guide sheath 512 having at least one lumen 514 for deployment of a recording element or recording element array alongside the shaft of the probe 510. To avoid interference, in the illustrated embodiment, the recording element/array may be extended only during periods of probe inactivity (or inactivity of those element(s) capable of interference with recording element/array) and/or extended only far enough to avoid interference during therapy (e.g., extended along the length of an internal sheath 516 of the probe 510). In other embodiments, the recording element is included within the outer tube of the probe design. For example, in a multi-lumen probe design such as the lumens 904 through 906 of FIG. 10 described in relation to the probe 900 of FIG. 9, a separate lumen may be provided for inclusion of the lesion detection element along an external surface of the probe (e.g., similar to the lumen 514 of the guide sheath 512). In positioning the recording element within a separate lumen, for example, the recording element 500 may be shielded in part from heating, cooling, and/or EMC effects.

As illustrated in FIG. 5C, the probe 510 is inserted into a sleeve 520 including multiple recording element rings 522. The sleeve 510, in some embodiments, is formed of flexible material, for example to snuggly wrap around the probe 510. The stretchiness of the sleeve 520, in some embodiments, may be selected to accept a range of probe diameters. In another example, the sleeve may be formed of stiffened material, shaped to surround the probe 510. The recording element rings 522 may each be connected to a recording element lead (e.g., wire) delivering signals recorded by the recording element rings 522 to an external device for analysis.

In some embodiments, the probe 510 may be extended and retracted within the sleeve 520. For example, when the probe 510 is inactive and signals collected by the recording element rings 522 are being monitored, the probe 510 may be retracted within the sleeve 520 such that the recording element rings 522 are deployed as close as possible to the tissue near a tip region 524 of the probe 510.

In some embodiments, a recording element/array may be included inside a probe. For example, the recording element/array may be included within a same lumen as one or more other elements of the probe, such as the temperature sensor 408 described in relation to FIG. 4. In another example, the recording element/array may be included in a separate lumen with an external access, such that the recording element/array may be deployed externally to the probe's outer sheath, and then retracted when not in use.

As illustrated in FIG. 5D, a probe 530 includes a port 532 in a side of a shaft section 534a. Turning to FIG. 5E, a recording element 536 (e.g., electrode) is extended through the port 532 along the side of a capsule section 534b of the probe 530 to utilize the features of the recording element 536. In this manner, the probe 530 may be positioned at the target tissue prior to deployment of the recording element 536. In some embodiments, the recording element 536 is configured for deployment short of an emission region 538 of the probe 530, for example to avoid interference between the recording element 536 and therapeutic emissions of the probe 530. In other embodiments, the recording element 536 may be extended within the emission region 538 or even beyond the tip of the probe 530. The recording element 536, in some implementations, includes an angled shape to allow deployment of the recording element 536 through the port 532 to a stop point. In other examples, the recording element 536 is a flexible electrode, such that it may be deployed at variable distances beyond the port 532. Although illustrated as a single port 532 and recording element 536, in other embodiments, the probe 530 may include multiple ports 532 and multiple recording elements 536. In further embodiments, the probe 530 may include a recording array deployed from the single port 532.

As illustrated in FIGS. 5F through 5H, in some implementations, a recording instrument 540 is designed for collecting and analyzing signals obtained by one or more recording elements, such as recording elements 542 and 543. The recording instrument 540, for example, may be designed with an outer diameter between 0.75 and 1.22 mm for intracranial placement. As shown in FIG. 5F, a first recording element contact surface 542a is disposed at a tip of the recording instrument 540, and a second recording element contact surface 543a is disposed along a shaft region of the recording instrument 540. Turning to FIG. 5G, a cross-sectional view 550 of the probe 540 illustrates a first recording element lead 542b and a second recording element lead 542b disposed along the shaft region of the recording instrument 540. The recording element leads 542b, 543b, for example, may connect to a connection unit 562. Further, signals supplied by the recording element leads 542b, 543b may be provided to an external analyzer, controller, and/or recording device via a lead connection 572 of the connection unit 562. Although described as a lead connection 572, in other embodiments, the connection unit 562 may be designed for wireless transmission of the recording element signals, for example via a Bluetooth, Wi-Fi, or other near field communication (NFC) connection to a wirelessly connected recording and/or analysis device.

The recording instrument 540, in some implementations, includes an outer tubing 552 of an insulating material, such as the signals recorded by the recording element contact surfaces 542a, 543a are isolated from each other. In a particular example, the outer tubing 552 is formed of a flexible polymer. The contact surfaces, for example, may be integrated with the flexible polymer material.

In some implementations, the recording instrument 540 includes a coolant supply tube 546 for delivery cooling fluid or gas to a cooling zone region 544 of the recording instrument 540 (as illustrated in FIG. 5F). The cooling fluid or gas, for example, may be introduced at a cooling gas input 568 of the connection unit 562. Further, the cooling gas (or expanded cooling fluid) may be exhausted from the recording instrument 540 via an exhaust chamber 554 disposed along the shaft of the recording instrument 540 (as illustrated in the cross-sectional view 550 of FIG. 5G) and out an gas exhaust port 570 of the connection unit 562. Alternatively, the gas exhaust port 570 may be replaced by a collector assembly for return collection and recycling of coolant.

The connection unit 562, in some implementations, includes a control module for regulating coolant pressure and/or flow rate to provide a desired temperature to the cooling zone region 544 of the recording instrument 540. The control module, for example, may monitor temperature within the cooling zone region 544 through temperature sensor signals supplied by a temperature sensor 548 (e.g., thermocouple) and modulate coolant feed accordingly to maintain a desired temperature or temperature modulation pattern. Additionally or alternatively, coolant pressure may be manually adjusted via a manual pressure control valve 566 of the connection unit 562.

In a particular example, the connection unit 562 may modulate temperatures of tissue within, in some examples, up to 3 mm, up to 5 mm, or up to 10 mm of the cooling zone region 544 of the recording instrument 540 by first cooling the tissues using the cooling gas (e.g., via JT cooling as discussed, for example, in relation to FIG. 4) and then allowing the tissue to warm. Rather than allowing the body to return to a baseline temperature, in some embodiments, the connection unit 562 may deploy thermal energy to the cooling zone region 544 to encourage warming of the tissues. In a particular example, the connection unit 562 may supply warming gas or fluid. In another illustrative example, the connection unit may alter gas pressure of the coolant to encourage increased temperature at the cooling zone region 544 of the recording instrument 540. The Joule-Thomson principle can be used to either warm or cool a gas expanding through a throttling device such as the cooling injection tube. Depending on the J-T inversion temperature, some gases may warm when expanded (e.g., Helium or hydrogen) and other gases cool (e.g., CO2, nitrogen, argon).

In some implementations, the connection unit 562 includes a temperature readout display 564. For example, the control module may translate temperature signals supplied by the temperature sensor 548 into digits for presentation upon the temperature readout display 564 for review by a medical professional.

Although illustrated as connecting to a single recording instrument 540, in other implementations, the connection unit 562 may be configured to provide input/output connections for two or more recording instruments 540. For example, between two and ten recording instruments 540 may be positioned at various locations within a patient, and the recording instruments 540 may be configured for thermal modulation control and/or analysis via the single connection unit 562.

FIG. 5I illustrates a flow chart of a method 580 for using interstitial signal recording elements, such as the recording elements described in relation to FIGS. 5A through 5G. The method 580, in some embodiments, may be performed at least in part by processing circuitry of an interstitial probe. In some embodiments, the method 580 may be performed at least in part upon processing circuitry separate from the device including the recording element, such as a controller in wired or wireless communication with the device including the recording element. In a particular embodiment, signals read by one or more recording elements may be collected by collection circuitry, and processing circuitry in wired or wireless communication with the collection circuitry may perform subsequent steps of the method 580. Other permutations are possible.

In some implementations, the method 580 begins with disposing at least one signal recording element proximate a target tissue (582). The tissue, in some examples, may include brain tissue, spinal tissue, or pericardial tissue. In some embodiments, the signal recording element is a separate device (or, optionally, a set of signal recording elements may be included within a single device). For example, as illustrated in FIGS. 5F through 5H, the recording instrument 540 may include one or more rings of recording elements (e.g., such as the rings 502 illustrated in on the probe 500 of FIG. 5A). In other embodiments, the signal recording element is coupled to an interstitial therapy instrument. FIG. 5B, for example, illustrates both a recording instrument and a probe deployed via a same guide sheath, while FIG. 5C illustrates a recording element sheath surrounding the probe. In further embodiments, the signal recording element is integrated into an interstitial therapy instrument. For example, as illustrated in FIGS. 5A 5D, and 5E the recording elements are integrated into the probes.

In some implementations, signal recordings are received from the signal recording element(s) (584). The signals may be recorded in a continuous or periodic manner. For example, signals may be recorded opposite an energy emission pattern of a probe deployed with (e.g., proximate to, coupled to, or integrated with) the signal recording element(s) such that the energy emission pattern is not disrupted by the signal recording element(s) or vice versa. The signals recordings may include discrete signal measurements and/or signal patterns sensed over a period of time. The signals, in some examples, may optionally be filtered, amplified, or otherwise adjusted prior to receipt by the method 580. Further, in some embodiments, the signal recordings may be provided with contemporaneous data such as, in some examples, time stamp data, tissue temperature recording data, probe temperature recording data, therapeutic emission pattern data, and/or biometric data (e.g., heart rate, pulse rate, breathing rate, etc.) of the patient.

In some implementations, the signal recordings are analyzed to detect an abnormal signal pattern (586). Analysis may include monitoring for abnormal signal patterns indicative of one or more of a brain tissue hibernation pattern, a brain tissue warm-up (e.g., post hibernation) pattern, a seizure activity pattern or pre-seizure activity pattern (e.g., epilepsy onset spot), a neurological symptom pattern or pre-symptom pattern (e.g., Parkinson's disease tremors), location of a lesion, location of a critical structure (e.g., artery, nerve, functional motor strip within motor cortex, corticospinal track, and/or other critical neural pathways, etc.), alterations in the blood brain barrier (e.g., disruption of the BBB), abnormal biorhythms, or other electrical activity markedly different from a baseline for the tissue region (e.g., brain, thoracic cavity, spinal region). In some embodiments, analysis includes coordinating signal data with additional brain activity measurement data, for example derived non-invasively through imaging or other means. For example, analyzing the signal recordings may include analyzing the signal recordings in light of magnetoencephalography (MEG) data or in an effort to confirm MEG data. Further, in some embodiments, analyzing the signal recordings includes analyzing the signal recordings in light of historic signal recording data. For example, detecting an abnormal signal pattern may include detecting movement from previously recorded abnormal signal pattern to a desirable (e.g., normal, healthy, or indicative of success of a therapeutic treatment) or baseline signal pattern.

In some implementations, if an abnormal signal pattern is detected (588) and the recording elements are part of an automated system for therapeutic treatment using an interstitial therapy instrument, an appropriate adjustment to at least one of an emissive output, a therapeutic profile, and a therapeutic instrument position is determined (592). In a first example, the abnormal signal pattern may identify position of a lesion, and adjustment of the emissive output may include delivery of treatment (e.g., thermal therapy, cryotherapy, and/or pharmacological therapy, etc.) to the lesion. In another example, the abnormal signal pattern may identify disruption of the blood brain barrier, and adjustment of therapeutic profile may include shortening a timeframe for delivery of a therapeutic treatment. In an example related to therapeutic instrument position, the abnormal signal pattern may be indicative of location of a critical structure, and the position (e.g., linear position, rotational position, etc.) may be adjusted to avoid damage to the critical structure. The examples are provided for illustrative purposes only, and are in no way mean to be limiting to the opportunities for automated response to detection of an abnormal signal pattern recorded by the signal recording element(s).

In some implementations, after determining the appropriate adjustment, the appropriate adjustment is effected (594). As presented in greater detail, for example, in U.S. Pat. No. 8,979,871 entitled “Image-Guided Therapy of a Tissue” and filed Mar. 15, 2013 (incorporated by reference herein in its entirety), a probe driver may be activated to adjust physical position of the thermal therapy instrument. In another example, as discussed in greater detail in relation to FIGS. 6A and 6B, emission output and/or emission patterns may be adjusted by the controller (e.g., onboard the probe or external thereto) of a thermal therapy, cryotherapy, or temperature modulation therapy instrument. Prior to effecting an adjustment, in some embodiments, the method 580 may prompt an operator for approval to effect the adjustment. For example, a visual and/or audible prompt may alert the operator to the option to effect the recommended adjustment. Effecting adjustment, further, may include prompting an operator to manually perform one or more adjustments.

In some embodiments, if the system is not designed for automated adjustment (590), an alert may be issued for attention of a system operator (596). For example, a visual output including a signal pattern display (e.g., graph, chart, or other illustration indicative of the received signal recordings) and/or signal pattern identifier (e.g., visual arrangement and/or text indicative of particular type of abnormal signal pattern) may be presented upon a display device provided for the system operator. In another example, an audible alert, such as a verbal message, a warning tone, or a series of intonations representative of an abnormal signal pattern may be output for the system operator via a speaker device.

Whether or not an abnormal signal pattern was detected 588, in some implementations, the method 580 proceeds to continue to receive signal recordings (584).

Although described as a particular series of steps, in other implementations, the method 580 may include more or fewer steps. For example, after disposing the signal recording element(s) proximate a target tissue (582) and prior to analyzing the signal recordings (586), the method 580 may receive additional data recordings (e.g., as described in relation to step 586) separate from the signal recordings (e.g., from one or more separate instruments or systems). In another example, after detecting the abnormal signal pattern (588), rather than issuing an alert or determining an adjustment, the method 580 may simply log the abnormal signal pattern (e.g., for later use as historic signal pattern data).

Additionally, in other implementations, steps of the method 580 may be performed in a different order. For example, the method 580 may initially deliver therapeutic output, then receive (584) and analyze (586) signal recordings in an effort to determine success of the delivered therapeutic emission. Other modifications of the method 580 are possible while remaining within the scope and purpose of the method 580.

FIGS. 13A-B, 14A-B, and 15A-C illustrate various embodiments of reduced profile probe designs. Reducing the profile of a probe is desirable for achieving minimally invasive surgery, performing surgical operations upon small bodies such as infants, juveniles and animals, and reaching otherwise difficult-to-reach in situ locations without negatively impacting surrounding tissues. A reduced profile probe, for example, can allow entry into small and narrow spaces in the brain while reducing patient injury. However, reducing the profile of a probe may adversely impact the cross-sectional area of a lumen inside the probe, as well as adversely impact thermal and mechanical properties as a result of reducing wall thickness of a probe shaft. The low profile probe, for example, may have a shaft substructure that has a reduced outside diameter to achieve the desired low profile for expanded lesion location and access while maintaining the desired properties (e.g., mechanical and/or thermal properties) to allow for the desired therapy procedures.

In some embodiments, single-layer low profile probe shafts are designed with a thermoplastic material for effecting thermal therapy by delivering energy to a targeted tissue area, such as brain tissue. In an exemplary embodiment, the energy modality is laser light and the thermal therapy is laser induced interstitial thermal therapy (LITT). The low profile probes described below, for example, may be used to effect reversible cellular damage and/or cellular death (ablation) as discussed above.

As discussed in further detail below, low profile probes may be configured with selected materials, lumen structures, and layer structures to provide desired and/or selected mechanical properties including straightness, rigidity, torque strength, column strength, tensile strength, kink resistance, and thermal properties such as thermal stability and thermal stress capacity. In some embodiments, the low profiled probes are MR-compatible. Maintaining the desired and/or selected mechanical and/or thermal properties, for example, can allow for remotely controlling and operating the low profile probe in a rotational and/or axial direction. In particular implementations, low profile probes are designed such that the tensile strength, torque strength, and column strength are greater than approximately 15N and the kink resistance is such that no damage to the probe results at curvature radiuses higher than approximately 40 mm. In some embodiments, the low profile probe shaft is air-tight to support modulated heating and cooling operations, such as the temperature-modulation probe designs and uses described above.

Low profile probe dimensions may vary, in some examples, based upon the style of the low profile probe (e.g., thermal therapy, cryotherapy, temperature modulation therapy), the anticipated probe deployment (e.g., intracranial, spinal, cardiac, etc.), the required thermal tolerances of the low profile probe, and/or the required structural tolerances of the probe (e.g., flexible vs. rigid). The wall thickness of the probe shaft, in particular, is related to the stiffness of the low profile probe, which helps the low profile probe stay on trajectory (or allows the probe to deflect therefrom). The following are examples of low profile probe dimensions. In a first example, the inner shaft of a low profile probe has an outer diameter of 2.0 mm, within a tolerance of 0.03 mm and an inner diameter of 1.5 mm, within a tolerance of 0.03 mm; in this embodiment, the sapphire lens has the same inner diameter. In a second example, the outer shaft of the low profile probe has an outer diameter of 2.25 mm, within a tolerance of 0.03 mm and an inner diameter of 2.07 mm, within a tolerance of 0.03 mm. In further examples, the shaft of various low profile probe designs may have an outer diameter of approximately 2.1 mm, approximately 2.2 mm, or less than approximately 3.2 mm. In additional examples, the shaft of various low profile probe designs may have an outer diameter of approximately 1.0 mm 1.2 mm, 1.5 mm, 1.7 mm, or 1.8 mm.

In some implementations, a low profile probe includes multiple internal lumens. The multi-lumen structure, for example, can provide a greater cross-sectional lumen area relative to the profile of the low profile probe while, at the same time, maintaining desired and/or selected mechanical properties including, for example, straightness, rigidity, torque strength, column strength, tensile strength, kink resistance, and thermal properties such as, for example, thermal stability and thermal stress capacity.

The multi-lumen structure, in some implementations, contains one or more thermal therapy-generating elements and one or more cryotherapy-generating elements for temperature modulation therapy. In addition, the multi-lumen structure may contain a temperature sensor, such as a thermocouple or fiber optic thermometer, to measure an initial (e.g., baseline) temperature of tissue prior to conducting temperature modulation therapy and/or to monitor internal temperatures during temperature modulation therapy. The thermal therapy-generating element, in a particular example, is a laser fiber. The laser fiber may be optionally selectively etched with a pattern to achieve a desired lasing pattern. Further to the particular example, the cryotherapy-generating element is a Joule-Thompson cooling apparatus. As discussed in greater detail above (e.g., in relation to FIGS. 6A, 6B, and 8), such a low profile temperature modulation probe may be designed to yield directional energy delivery (e.g., directional heating) or symmetric energy delivery (e.g., symmetric heating). Directional energy delivery, for example, may be achieved by a varying relative temperature while activating both the thermal therapy-generating element and the cryotherapy-generating element, by pulsing (e.g., turning on and off) the thermal therapy-generating element while maintaining activation of the cryotherapy-generating element, and/or by pulsing the cryotherapy-generating element while maintaining activation of the thermal therapy-generating element.

In some embodiments of a laser-based low profile temperature modulation probe designed for LITT, a sapphire lens is utilized for the optimal laser transparency, and robust thermal (e.g., hot and cold) stress capacity. The multi-lumen structure of the low profile probe, in this example, provides comparable modulated lasing and cooling ability when compared with a probe having a larger profile and/or a single lumen.

Turning to FIG. 13A, a shaft 1302 of an example low profile probe 1300 includes a multi-lumen rod 1318 (internal cross-sectional detail illustrated in FIG. 13B). The rod 1318, for example, may be composed of a ceramic material selected for strength and break resistance. FIG. 13B illustrates two lumens 1310 defined along the length of the rod 1318, however, it should be understood that more than two lumens may be included in the rod 1318 depending on application. FIG. 13B depicts lumen 1310a as being larger than lumen 1310b, but it should be understood that both lumens 1310 can be approximately the same size. Lumen 1310a may contain, for example, a laser fiber 1312 which delivers laser energy, as well as a thermocouple 1314 (wire or fiber). The thermocouple 1314 may be shrink-wrapped within the lumen 1310a.

Lumen 1310a, in some embodiments, acts as a venting port for a cooling gas (or evaporated cooling liquid) that is delivered via lumen 1310b. A flexible cooling line, for example, may be connected to the distal end of the rod 1318 (not illustrated) and high pressure gas may flow through lumen 1310b, expanding in a tip region 1304 of the low profile probe 1300, and then flowing back through lumen 1310a.

As illustrated in FIG. 13B, the rod 1318, in some embodiments, is covered by a thin-walled polyether ether ketone (PEEK) plastic tube 1316. The PEEK tube 1316, for example, may act as a protective barrier in case the rod 1318 breaks. In the result of a break, the PEEK plastic tube 1316 will keep the probe shaft 1302 connected so it can be completely removed from the patient. Breakage of the rod 1318 may be detected by the low profile probe 1300 and/or a controller thereof, in some examples, based upon unanticipated fluctuations in cooling gas pressure and/or the probe tip temperature. To guard against breakage, material properties and burst pressure ratings of the materials of the rod 1318 may be selected to conform to thermal operating ranges of the particular probe design. Further, in manufacture, the thermal operating ranges of the materials may be verified/validated to prevent breakage or coolant gas leakage during operation. PEEK plastic material is biocompatible which makes it an acceptable material for contact with the patient.

Extending from and integral with the tip 1304 of the low profile probe 1300, in some embodiments, is a lens 1306. The lens 1306, for example, may be composed of machined sapphire. As illustrated, the lens 1306 is bonded to the proximal end of the rod 1318 and is also inserted into the end of the PEEK plastic tube 1316. The energy delivery occurs via the lens 1306. As illustrated, the tip 1304 of the lens 1306 has a torpedo-like nose shape. In another embodiment (not illustrated), the tip 1304 of the lens 1306 has a rounded nose shape.

As discussed above, in the example embodiment illustrated, the components of the probe shaft 1302 are composed of heterogeneous materials (e.g., PEEK and ceramic). In other embodiments, homogenous materials form the components of the probe shaft 1302. Each material may be selected to achieve the desired and/or selected mechanical and/or thermal properties for the low profile probe 1300. In some embodiments, the shaft 1302 and the PEEK tube 1316 are bonded together.

Turning to FIGS. 14A and 14B, a shaft 1402 of a low profile probe 1400 is shown as including a multi-lumen structure (illustrated in a cross-sectional view of the shaft 1402 in FIG. 14B). The shaft 1402, for example, may be configured as a PEEK rod. The style and sizes of the lumens 1410, in some embodiments, are similar to the lumens 1310 of FIG. 13B. To create the low profile probe 1400, in one example, the proximal end of the shaft 1402 may be drilled to allow the distal end of a lens 1406 (e.g., machined sapphire, etc.) to insert and bond. The lens 1406, in this example, may be manufactured with an outer diameter in accordance with the outer and/or inner diameter of the probe shaft 1402. In other embodiments (not illustrated), the probe shaft 1402 is composed of two PEEK rods (i.e., inner and outer shafts).

FIGS. 15B and 15C illustrate an example multi-layer single lumen (e.g., open cylinder) probe shaft design of a shaft 1502 of a low profile probe 1500 illustrated in FIG. 15A. As illustrated in FIG. 15C, the shaft 1502 includes an inner layer 1506a and an outer layer 1506b. In other embodiments, a multilayer shaft structure includes three or more layers. The layers may be composed of different or same materials. For example, each of the layers 1506 may be composed of a thermoplastic selected for having a high Young's modulus value. In additional examples, one or more layers may be composed of a non-solid surface material such as, in some examples, a coiled or braided structure. Materials useful in manufacturing the non-solid surface layer, in some examples, include PTFE, PEEK, Polyimide, Polyamide, Polyethylene and PEBAX. In some embodiments, a layer may include winding material made of stainless steel, Nitinol, Nylon or PEEK. Example layers for a probe designed to bend away from skull include a polyimide layer for rigidity (e.g., for a distal portion of a probe shaft) and a PTFE layer for flexibility (e.g., for a proximal portion of a probe shaft,). Other multi-layer probe shaft designs may include a polyimide layer disposed under an etched layer of PTFE for improved bonding characteristics. As illustrated, the inner layer 1506a is thicker than the outer layer 1506b. In other embodiments, the layers may be all of the same thickness, or an inner layer may be thinner than an outer layer. For overall dimensions, in some examples, the design of the shaft 1502 may allow for an outer diameter of a lens 1504 of the probe 1500 to be reduced to about 2.2 mm.

The layers 1506 of the shaft 1502, as illustrated in FIG. 15B, are linearly aligned to create a counterbore 1508 in the proximal end of the shaft 1502. During manufacture of the probe 1500, for example, the distal end of the lens 1504 may be glued to the proximal end of the shaft 1502 at the counterbore 1508. The proximal end of the shaft 1502, as illustrated in FIG. 15B, can be structured such that the lens 1504 can be configured to have both lap join and bud join to the shaft 1502 for secured bond strength. The length of the counterbore, in one example, is controlled match the shoulder length of the lens 1504. In a particular example, the design of the probe 1500 may allow for reduction of a shoulder diameter of the lens 1504 to approximately 2.0 mm.

Turning to FIG. 9, an example laser probe 900 includes a fiber 901 which extends from a tip portion 902 including a light dispersion arrangement connected to a suitable light source at an opposed end of the fiber 901. The light dispersion arrangement, for example, may include a light-directing element at an end of the fiber 901 for directing the light from the laser to the predetermined direction relative to the fiber 901 forming the limited angular orientation within a disk surrounding the axis of the probe 900. The probe 900 further includes, in some embodiments, a support tube 903 in the form of a multi-lumen catheter for the fiber 901 which extends along the fiber 901 from an end 904 of the tube just short of the tip 902 through to a position beyond a fiber drive system configured for controlling the orientation of the fiber within the patient. The fiber drive system, in one example may include a drive motor supported in fixed adjustable position on a stereotaxic frame. The motor may communicate through a control line to a device controller. In general the device controller may receive information from an imaging console such as an MRI console and from position detectors of the motor. The device controller may use this information to control the motor and to operate a power output from the laser, thereby controlling the position and amount of heat energy applied to the part within the body of the patient.

The support tube 903, as illustrated in FIG. 10, includes a first cylindrical duct 904 extending through the tube and two further ducts 905 and 906 parallel to the first duct 904 and arranged within a cylindrical outer surface 907 of the support tube 903.

Returning to FIG. 9, the supporting tube 903 has at its end opposite the outer end 904 a coupling 908 which is coupled to (e.g., molded onto, integrated into, etc.) the end 909 and connects individual supply tubes 910, 911 and 912 each connected to a respective one of the ducts 904, 905 and 906 of FIG. 10. Multi-lumen catheters of this type are commercially available and can be extruded from suitable material to provide the required dimensions and physical characteristics. Thus the duct 904 is dimensioned to closely receive the outside diameter of the fiber 901 so that the fiber 901 can be fed through the duct tube 910 into the duct 904 and can slide through the support tube 903 until the tip 902 is exposed at the end 904.

While tubing may be available which provides the required dimensions and rigidity, in many cases, the tubing is however flexible so that it bends side to side and also will torsionally twist. The support tube 903, in some embodiments, is therefore mounted within an optional stiffening tube or sleeve 914, which extends from an end 915 remote from the tip 902 to a second end 916 adjacent to the tip 902. The second end 916 is however spaced rearwardly from the end 904 of the support tube 903, which in turn is spaced from the tip 902. The distance from the second end 916 to the tip 902 may be arranged to be less than a length of the order of 1 inch. The stiffening tube 914 may be formed of a suitable stiff material that is non-ferro-magnetic so that it is MRI compatible. The support tube 903 may be bonded within the stiffening tube 914 so that it cannot rotate within the stiffening tube 914 and cannot move side to side within the stiffening tube 914. The stiffening tube 914, in some embodiments, is manufactured from titanium, ceramic or other material that can accommodate the magnetic fields of MRI or be similarly compatible with other forms of imaging or detecting means relevant to the use. Titanium generates an artifact within the MRI image. For this reason, the second end 916 may spaced as far as practicable from the tip 902 so that the artifact is removed from the tip 902 to allow proper imagining of the tissues.

In some embodiments, a capsule 920 in the form of a sleeve 921 and domed or pointed end 922 is provided at the second end 916 of the stiffening tube 914. The sleeve 921 may surround the second end 916 of the stiffening tube 914 and be bonded thereto so as to provide a sealed enclosure around the exposed part of the support tube 903. The capsule 920, in some embodiments, is formed of quartz crystal so as to be transparent to allow the escape of the disbursed light energy from the tip 902. The distance of the end of the stiffening tube 914 from the tip 902 may be arranged such that the required length of the capsule 920 does not exceed what can be reasonably manufactured in the transparent material required.

In some embodiments, supply tube 911 is connected to a supply 925 of a cooling fluid and the supply tube 912 is connected to a return collection 926 for the cooling fluid. Thus, the cooling fluid is pumped through the duct 905 and escapes from the end 904 of the support tube 903 into the capsule 920 and then is returned through the duct 906. The cooling fluid can simply be liquid nitrogen allowed to expand to nitrogen gas at cryogenic temperatures and then pumped through the duct 905 and returned through the duct 906 where it can be simply released to atmosphere at the return 926.

In other embodiments, the supply 925 and the return 926 form parts of a refrigeration cycle where a suitable coolant is compressed and condensed at the supply end and is evaporated at the cooling zone at the capsule 920 so as to transfer heat from the tissue surrounding the capsule 920 to the cooling section at the supply end.

The arrangement set forth above allows the effective supply of the cooling fluid in gaseous or liquid form through the ducts 905 and 906 and also effectively supports the fiber 901 so that it is held against side to side or rotational movement relative to the stiffening tube 914. The location of the tip 902 of the fiber 901 is therefore closely controlled relative to the stiffening tube 914. In some embodiments, the stiffening tube 914 is driven by couplings 930 and 931, shown schematically in FIG. 9, of the type driven by reciprocating motor arrangements as set forth in U.S. Pat. No. 7,167,741 to Torchia, entitled “Hyperthermia Treatment and Probe Therefore” and filed Dec. 14, 2001, incorporated herein by reference in its entirety.

Turning to FIGS. 11 and 12, an example tip section of an alternative probe is illustrated, in which cooling of the tip section is effected using expansion of a gas into an expansion zone. The tip only is shown as the remainder of the probe and its movements are substantially as previously described.

In some embodiments, the probe includes a rigid extruded tube 1100 of a suitable material, for example titanium, that is compatible with MRI (non-ferromagnetic) and suitable for invasive medical treatment. The probe further includes a smaller cooling fluid supply tube 1102 which may be separately formed, for example by extrusion, and may be attached by adhesive to the inside surface of the outer tube 1100. An optical fiber 1104 is also attached by adhesive to the inside surface the outer tube 1100 so that the fiber 1104 is preferably diametrically opposed to the cooling supply tube 1102.

The cooling supply tube 1102 is swaged at its end to form a neck section 1105, which projects beyond the end of the tube 1101, to form a neck section of reduced diameter at the immediate end of the tube 1102. Thus in manufacture the extruded tube 1101 may be cut to length so as to define a tip end 1107 at which the outer tube terminates in a radial plane. At the tip end 1107 beyond the radial plane, the outer of the inner tube 1102 may be swaged by a suitable tool so as to form the neck section 1105 having an internal diameter, for example, of the order of 0.003 to 0.005 inch.

The fiber 1104, in some embodiments, is attached to the tube 1101 so that a tip portion 1108 of the fiber 1104 projects beyond the tip end 1107 to a chamfered end face 1109 of the fiber. As illustrated, the chamfered end face 1109 is cut at approximately 45 degrees to define a reflective end plane of the fiber 1104.

The tip end 1107, in some embodiments, is covered and encased by an end cap 1110 (e.g., molded quartz) that includes a sleeve portion 1111 closely surrounding the last part of the tube 1100 and extending beyond the tip end 1107 to an end face 1112, which closes the capsule. The end face 1112 is tapered to define a nose 1113, which allows the insertion of the probe to a required location. The end of the tube 1101 may be reduced in diameter so that the capsule has an outer diameter matching that of the main portion of the tube 1101. However in the arrangement shown in FIG. 11 the capsule is formed on the outer surface so that its outer diameter is larger than that of the tube and its inner diameter is approximately equal to the outer diameter of the tube.

A temperature sensor 1114 (e.g., thermocouple, fiber optic thermometer, etc.), in some embodiments, is attached to the inside surface of the outer tube 1100 at the tip end 1107 and includes connecting wires 1115 which extend from the temperature sensor 1114 to a control unit 1126. Thus the temperature sensor 1114 provides a sensor to generate an indication of the temperature at the tip end 1107 within the capsule. A fiber optic thermometer, in one example, may provide the benefit of being fully immune to RF environments and therefore require no electromagnetic compatibility (EMC) filtering. The capsule may be welded to or bonded to the outer surface of the tube as indicated at 1118 so as to form a closed expansion chamber within the capsule beyond the tip end 1107. In some embodiments, an inner surface 1116 of the capsule is of the same diameter as the outer surface of the tube 1100 so that the expansion chamber beyond the end of the tube 1100 has the same exterior dimension as the tube 1100.

The capsule, in some embodiments, is transparent so as to allow the reflected beam of the laser light from the end face 1109 of the fiber 1104 to escape through the transparent capsule in the limited angular direction substantially at right angles to the longitudinal axis of the fiber 1104 and within the axial plane defined by that longitudinal axis.

The tube 1102, in some embodiments, is connected at its end opposite to the neck section 1105 to a coolant supply 1119, which forms a pressurized supply of a suitable cooling fluid or gas such as carbon dioxide or nitrous oxide. The coolant supply 1119, in some embodiments, is controlled by the control unit 1126 to generate a predetermined pressure within the fluid supply to the supply tube 1102 which can be varied so as to vary the flow rate of the fluid through the neck section 1105. The fluid may be supplied at normal or room temperature without cooling. The fluid, in some embodiments, is a gas at this pressure and temperature but fluids that are liquid can also be used provided that they form a gas at the pressures within the expansion chamber and thus go through an adiabatic gas expansion through the restricted orifice into the expansion chamber to provide the cooling effect.

Thus the restricted orifice has a cross-sectional area very much less than that of the expansion chamber and the return duct provided by the inside of the tube 1101. The items that reduce the effective cross-sectional area of the return tube 1101 may include, in some examples, the optical fiber 1104, the supply tube 1102, the thermocouple wires 1115, a shrink tube that fixes the thermocouple wires 1115 to the optical fiber 1104, and/or adhesives used to bond the items into place (e.g., at the inlet of the discharge duct).

Without the area of the adhesives included in the calculation, in some embodiments the exhaust duct area is about 300 times larger than a target size of the delivery orifice diameter (e.g., about 0.004″). When considering the area occupied by the adhesives, the exhaust duct inlet area may be approximately 200 to 250 times larger than the delivery orifice diameter. Considering the manufacturing tolerance range of the supply tube orifice diameter alone, the exhaust duct area may be anywhere between 190 to 540 times larger than the orifice area (without considering the area occupied by adhesives). Therefore, it is estimated that about a 200/1 gas expansion may be required to achieve appropriate cooling. This may allow the gas as it passes into the expansion chamber beyond the neck section 1105, in the particular example, to expand as a gas thus cooling the capsule and the interior thereof at the expansion chamber to a temperature in the range of approximately −20 C to 0 C. This range has been found to be suitable to provide the required level of cooling to the surface of the capsule so as to extract heat from the surrounding tissue at a required rate. Variations in the temperature in the above range can be achieved by varying the pressure from the coolant supply 1119 so that, in one example, the pressure would be of the order of 700 to 850 psi at a flow rate of the order of 5 liters per min.

The tube 1102, in some embodiments, has an outside diameter of the order of 0.014 inch OD, while a tube 1103 has a diameter of the order of 0.079 inch. Thus a discharge duct for the gas from the expansion chamber is defined by the inside surface of the tube 1100 having a flow area which is defined by the area of the tube 1100 minus the area taken up by the tube 1102 and the fiber 1104. This allows discharge of the gas from the expansion chamber defined within the capsule at a pressure of the order of 50 psi so that the gas can be simply discharged to atmosphere if inert or can be discharged to an extraction system or can be collected for cooling and returned to the coolant supply 1119 if economically desirable. Tip cooling may be necessary, in certain uses, for optimum tissue penetration of the laser or heating energy, reduction of tissue charring and definition of the shape of the coagulated zone. The gas expansion thus provides an arrangement that is suitable for higher power densities required in this probe to accommodate the energy supplied by the laser heating system.

The tip portion 1108 of the fiber 1104, in some embodiments, is accurately located within the expansion zone since it is maintained in fixed position within the capsule by its attachment to the inside surface of the outer tube 1100. The fiber 1104 may be located forwardly of the tip end 1107 sufficiently that the MRI artifact generated by the tip end 1107 is sufficiently removed from the plane of the fiber tip portion 1108 to avoid difficulties in monitoring the temperature within the plane of the fiber tip portion 1108. The outlet orifice of the tube 1102 may also be located forwardly of the tip end 1107 so as to be located with the cooling effect generated thereby at the plane of the fiber tip portion 1108 or end face 1109 thereof.

The end face 1109, in some embodiments, is located within the expansion chamber so that it is surrounded by the gas with no liquid within the expansion chamber. Thus, in practice there is no condensate on the end face 1109 nor any other liquid materials within the expansion chamber that would otherwise interfere with the reflective characteristics of the end face 1109.

The end face 1109, in some embodiments, is coated with a reflective coating such as a dual dielectric film. This may provide a reflection at the two wavelengths of the laser light used as a visible guide beam and as the heat energy source, such as He—Ne and Nd:YAG respectively. An alternative coating is gold, which can alone provide the reflections at the two wavelengths.

The arrangement of the probe of FIGS. 11 and 12 provides excellent MRI compatibility both for anatomic imaging as well as MR thermal profiling. Those skilled in the art will appreciate that the cooling system in accordance with the above description may also be used with circumferential fibers having point-of-source energy.

In some embodiments, in operation, the temperature within the expansion zone is monitored by the temperature sensor 1114 so as to maintain that temperature at a predetermined temperature level in relation to the amount of heat energy supplied through the fiber 1104. Thus the pressure within the fluid supply is varied to maintain the temperature at that predetermined set level during the hyperthermic process.

As described previously, the probe may moved to an axial location within the volume to be treated and the probe may rotated in steps so as to turn the heating zone generated by the beam B into each of a plurality of segments within the disk or radial plane surrounding the end face 1109. Within each segment of the radial plane, heat energy is supplied by the beam B that is transmitted through the capsule into the tissue at that segment. The heat energy is dissipated from that segment both by reflection of the light energy into adjacent tissue and by conduction of heat from the heated tissue to surrounding tissue. As stated previously, those skilled in the art will appreciate that the probe used with the cooling system in accordance with the description above may include circumferential fibers having point-of-source energy.

The surface of the capsule, in some embodiments, is cooled to a temperature so that it acts to extract heat from the surrounding tissue at a rate approximately equal to the dissipation or transfer of heat from the segment into the surrounding tissue. Thus the net result of the heating effect is that the segment alone is heated and surrounding tissue not in the segment required to be heated is maintained without any effective heating thereon, that is no heating to a temperature which causes coagulation or which could otherwise interfere with the transmission of heat when it comes time to heat that tissue in another of the segments. In this way when a first segment is heated to the required hyperthermic temperature throughout its extent from the probe to the peripheral surface of the volume, the remaining tissues in the areas surrounding the probe are effectively unheated so that no charring or coagulation has occurred which would otherwise prevent dissipation of heat and in extreme cases completely prevent penetration of the beam B.

Thus when each segment in turn has been heated, the probe can be rotated to the next segment or to another segment within the same radial plane and further heating can be effected of that segment only.

In practice in one example, the laser energy can be of the order of 12 to 15 watts penetrating into a segment having an angle of the order of 60 to 80 degrees to a depth of the order of 1.5 cm. In order to achieve this penetration without causing heating to the remaining portions of the tissue not in the segment, cooling of the outside of the capsule to a temperature of the order of −5° C. may be required.

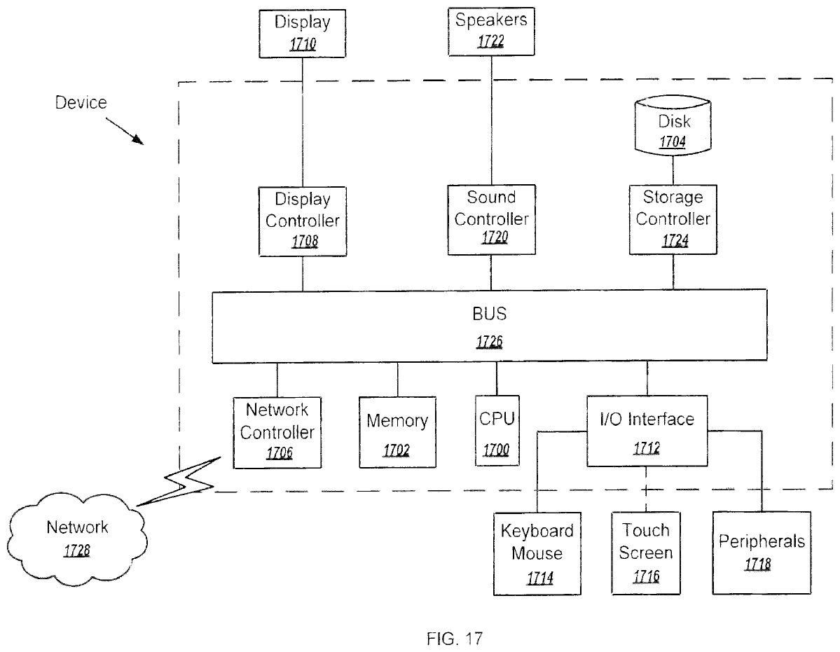

Next, a hardware description of the computing device, mobile computing device, or server according to exemplary embodiments is described with reference to FIG. 17. In FIG. 17, the computing device, mobile computing device, or server includes a CPU 1700 which performs the processes described above. The process data and instructions may be stored in memory 1702. These processes and instructions may also be stored on a storage medium disk 1704 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device, mobile computing device, or server communicates, such as a server or computer.

Further, a portion of the claimed advancements may be provided as a utility application, background daemon, or component of an operating system, or combination thereof, executing in conjunction with CPU 1700 and an operating system such as Microsoft Windows 7 or 8, UNIX, Solaris, LINUX, Apple MAC-OS and other systems known to those skilled in the art.

CPU 1700 may be a Xenon or Core processor from Intel of America or an Opteron processor from AMD of America, or may be other processor types that would be recognized by one of ordinary skill in the art. Alternatively, the CPU 1700 may be implemented on an FPGA, ASIC, PLD or using discrete logic circuits, as one of ordinary skill in the art would recognize. Further, CPU 1700 may be implemented as multiple processors cooperatively working in parallel to perform the instructions of the inventive processes described above.

The computing device, mobile computing device, or server in FIG. 17 also includes a network controller 1706, such as an Intel Ethernet PRO network interface card from Intel Corporation of America, for interfacing with network 1728. As can be appreciated, the network 1728 can be a public network, such as the Internet, or a private network such as an LAN or WAN network, or any combination thereof and can also include PSTN or ISDN sub-networks. The network 1728 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The computing device, mobile computing device, or server further includes a display controller 1708, such as a NVIDIA GeForce GTX or Quadro graphics adaptor from NVIDIA Corporation of America for interfacing with display 1710, such as a Hewlett Packard HPL2445w LCD monitor. A general purpose I/O interface 1712 interfaces with a keyboard and/or mouse 1714 as well as a touch screen panel 1716 on or separate from display 1710. General purpose I/O interface also connects to a variety of peripherals 1718 including printers and scanners, such as an OfficeJet or DeskJet from Hewlett Packard.

A sound controller 1720 is also provided in the computing device, mobile computing device, or server, such as Sound Blaster X-Fi Titanium from Creative, to interface with speakers/microphone 1722 thereby providing sounds and/or music.

The general purpose storage controller 1724 connects the storage medium disk 1704 with communication bus 1726, which may be an ISA, EISA, VESA, PCI, or similar, for interconnecting all of the components of the computing device, mobile computing device, or server. A description of the general features and functionality of the display 1710, keyboard and/or mouse 1714, as well as the display controller 1708, storage controller 1724, network controller 1706, sound controller 1720, and general purpose I/O interface 1712 is omitted herein for brevity as these features are known.

One or more processors can be utilized to implement various functions and/or algorithms described herein, unless explicitly stated otherwise. Additionally, any functions and/or algorithms described herein, unless explicitly stated otherwise, can be performed upon one or more virtual processors, for example on one or more physical computing systems such as a computer farm or a cloud drive.

Reference has been made to flowchart illustrations and block diagrams of methods, systems and computer program products according to implementations of this disclosure. Aspects thereof are implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes and algorithms described herein may be performed in hardware or software executed by hardware, including computer processors and/or programmable circuits configured to execute program code and/or computer instructions to execute the functions, processes and algorithms described herein. Additionally, some implementations may be performed on modules or hardware not identical to those described. Accordingly, other implementations are within the scope that may be claimed.

1. A method for applying therapy using an interstitial probe, comprising:
positioning the interstitial probe proximate a target tissue, the interstitial probe comprising
a shaft region,
a tip region,
a temperature sensor,
at least one thermal therapy-generating element for thermal therapy emission via the tip region, and
at least one cryotherapy-generating element for cryogenic therapy emission via the tip region;
determining, by processing circuitry using the temperature sensor, an initial temperature of at least one of a) tissue proximate the tip region and b) the tip region;
identifying, by the processing circuitry, a therapeutic output for causing a temperature-induced effect to the target tissue, the therapeutic output comprising i) a thermal therapy emission of the thermal therapy-generating element, and ii) a cryogenic therapy emission of the cryogenic therapy element;
activating, by the processing circuitry, the therapeutic output by the interstitial probe;
during therapeutic output, monitoring, by the processing circuitry, temperatures collected by the temperature sensor relative to the initial temperature; and
based at least in part upon the monitoring, adjusting, by the processing circuitry, the therapeutic output.

2. The method of claim 1, wherein identifying the therapeutic output comprises identifying a modulation pattern, comprising
at least one higher thermal output corresponding to activation of a first thermal therapy element of the at least the thermal therapy-generating element for a first time interval, and
at least one lower thermal output corresponding to activation of a first cryogenic therapy element of the at least one cryogenic therapy element for a second time interval different than the first time interval.

3. The method of claim 1, further comprising receiving a thermal dose for effecting thermal therapy treatment, wherein identifying the therapeutic output comprises identifying the therapeutic output based at least in part on the thermal dose.

4. The method of claim 1, wherein identifying the therapeutic output comprises identifying the therapeutic output based at least in part on a desired effect upon the tissue, wherein the desired effect comprises at least one of altering normal biological function, altering abnormal biological function, and disrupting a blood-brain barrier.

5. The method of claim 4, wherein the desired effect enables at least one of delivery, speed, and efficacy of a secondary treatment to be applied at the region of interest, wherein the type of secondary treatment comprises at least one of a drug treatment, a chemical treatment, a biochemical treatment, and a radiation treatment.

6. The method of claim 5, wherein the secondary treatment comprises a delayed secondary treatment applied during a time period at least three hours after concluding therapeutic output.

7. The method of claim 6, wherein the secondary treatment comprises a series of at least two treatments, a second treatment of the at least two treatments delivered during a second time period at least three days after concluding therapeutic output.

8. The method of claim 5, wherein the secondary treatment comprises an intravenous drug treatment.

9. The method of claim 1, wherein the interstitial probe comprises at least a portion of the processing circuitry.

10. A system comprising:
an interstitial probe, comprising:
a shaft region,
a tip region,
a recording element disposed proximate the tip region,
at least one thermal therapy-generating element for thermal therapy emission via the tip region, and
at least one cryotherapy-generating element for cryogenic therapy emission via the tip region;
processing circuitry; and
a memory having instructions stored thereon, wherein the instructions, when executed by the processing circuitry, cause the processing circuitry to
identify a therapeutic output for causing a temperature-induced effect to a target tissue, the therapeutic output comprising at least one of i) a thermal therapy emission of the thermal therapy-generating element, and ii) a cryogenic therapy emission of the cryogenic therapy element;
activate the therapeutic output by the interstitial probe;
during therapeutic output, monitor data collected by the recording element; and
based upon the monitoring, identify, by the processing circuitry, at least one location responding to the therapeutic output with an abnormal signal pattern indicative of at least one of a physical state of the brain tissue and a medical condition.

11. The system of claim 10, wherein the instructions, when executed by the processing circuitry, further cause the processing circuitry to, responsive to identifying the at least one location:
select a second therapeutic output; and
apply the second therapeutic output at the at least one location.

12. The system of claim 11, wherein the second therapeutic output is selected to suppress a symptom pattern or pre-symptom pattern.

13. The system of claim 10, wherein the physical state of the brain tissue is a hibernation state.

14. The system of claim 10, wherein the abnormal signal pattern comprises one of a seizure activity pattern, a pre-seizure activity pattern, a neurological symptom pattern, and a neurological pre-symptom pattern.

15. The system of claim 10, wherein the recording element comprises at least one of an electroencephalography (EEG) and a stereo EEG (SEEG) recording element.

16. The system of claim 10, wherein the instructions, when executed by the processing circuitry, further cause the processing circuitry to, responsive to identifying the at least one location, present, for review by an operator of the system at a display device, information regarding the at least one of the physical state of the brain tissue and the medical condition.

17. A non-transitory computer readable medium having instructions stored thereon, wherein the instructions, when executed by processing circuitry, cause the processing circuitry to:
identify a therapeutic output for causing a temperature-induced effect to a target tissue, the therapeutic output comprising at least one of a thermal therapy emission of at least one of a thermal therapy-generating element, and a cryogenic therapy emission of a cryogenic therapy element;
activate the therapeutic output by an interstitial probe comprising at least one of the thermal therapy-generating element and the cryogenic therapy element, and a recording element;
during therapeutic output, monitor data collected by the recording element;
based upon the monitoring, identify, by the processing circuitry, at least one location responding to the therapeutic output with an abnormal signal pattern indicative of at least one of a physical state of the brain tissue and a medical condition;
responsive to identifying the at least one location, select a second therapeutic output comprising at least one of a second thermal therapy emission different than the thermal therapy emission, and a second cryogenic therapy emission different than the cryogenic therapy emission; and
apply the second therapeutic output at the at least one location while monitoring image data collected by the imaging system to identify suppression of the abnormal signal pattern.

18. The computer readable medium of claim 17, wherein the instructions, when executed by the processing circuitry, cause the processing circuitry to, prior to applying the second therapeutic output, receive, via an input device in communication with the processing circuitry, input submitted by an operator authorizing application of the second therapeutic output.

19.-20. (canceled)
